SEQUENCE LISTING (17 sequences)

SEQ ID NO: 1 - DNA, 47 bp, Homo sapiens
  5′clip (1)..(1): FAM = fluorescein
ctctcgggacgacatggattttccatcaacgaagtgcgtccgtcccg

SEQ ID NO: 2 - DNA, 13 bp, Homo sapiens
  3′clip (13)..(13): BHQ1 = Black Hole quencher 1
gtcgtcccgagag

SEQ ID NO: 3 - DNA, 44 bp, Homo sapiens
  5′clip (1)..(1): FAM = fluorescein
ctctctcgggacgctgggttttcccaggacgaagtccgtcccga

SEQ ID NO: 4 - DNA, 12 bp, Homo sapiens
  3′clip (12)..(12): BHQ1 = Black Hole quencher 1
cgtcccgagaga

SEQ ID NO: 5 - DNA, 44 bp, Homo sapiens
  5′clip (1)..(1): FAM = fluorescein
ctgctctcgggacgtggattttccgcatacgaagttgtcccgag

SEQ ID NO: 6 - DNA, 12 bp, Homo sapiens
  3′clip (12)..(12): BHQ1 = Black Hole quencher 1
gtcccgagagca

SEQ ID NO: 7 - DNA, 94 bp, Homo sapiens
gcgcataccagcttattcaattacgctcaggactaaaaatgggcgtgaccatgagctcagtatgggcgttacagatagtaagtgcaatctcggc

SEQ ID NO: 8 - DNA, 94 bp, Homo sapiens
gcgcataccagcttattcaatttcaaaggctgaccggatgggcttcgtgaccgggtacgccactgcctcgtgagatagtaagtgcaatctcggc

SEQ ID NO: 9 - DNA, 92 bp, Homo sapiens
gcgcataccagcttattcaattgatccatacaagggtaggttgacttccgcccaagggggcgaggccgtgagatagtaagtgcaatctcggc

SEQ ID NO: 10 - DNA, 93 bp, Homo sapiens
gcgcataccagcttattcaattaggaacgcacccgagtgttgcgcgagccagtggctggttggcccgtataagatagtaagtgcaatctcggc

SEQ ID NO: 11 - DNA, 94 bp, Homo sapiens
gcgcataccagcttattcaattgcgtacctaggatttactttctcaagatatttggagtgacaagtggcataagatagtaagtgcaatctcggc

SEQ ID NO: 12 - DNA, 94 bp, Homo sapiens
gcgcataccagcttattcaattgcatagtcatgcgggaatagttttcgatgagggctattggaggccccagcagatagtaagtgcaatctcggc

SEQ ID NO: 13 - DNA, 94 bp, Homo sapiens
gcgcataccagcttattcaattaacgtgtagaggtgttcacgcaccacgccgactgtggttgacccacagttagatagtaagtgcaatctcggc

SEQ ID NO: 14 - DNA, 94 bp, Homo sapiens
gcgcataccagcttattcaattcggtacgtgacgcaaaaaaggtcgggcggctgacatggcacctcgtctaaagatagtaagtgcaatctcggc

SEQ ID NO: 15 - DNA, 94 bp, Homo sapiens
gcgcataccagcttattcaattcggatagacaacatggggaagaacagtgtctagtcgtgggttgctctgtaagatagtaagtgcaatctcggc

SEQ ID NO: 16 - DNA, 94 bp, Homo sapiens
gcgcataccagcttattcaattcggtagtgcagcggcttgaaatcagccgtgctttcgggacggaactactcagatagtaagtgcaatctcggc

SEQ ID NO: 17 - DNA, 20 bp, Homo sapiens
tttttttttttttttttttt

(19) United States
(12) Patent Application Publication
(10) Pub. No.: US 2018/0371460 A1
(43) Pub. Date: Dec. 27, 2018

(54) Depletion of Abundant Serum Proteins to Facilitate Biomarker Discovery

(71) Applicants: UNIVERSITY OF UTAH RESEARCH FOUNDATION, Salt Lake City, UT (US); SONATA BIOSCIENCES, INC., Auburn, CA (US)

(72) Inventors: Jennifer Heemstra, Salt Lake City, UT (US); Frank Jahnke, Auburn, CA (US); Amberlyn Peterson, Auburn, CA (US)

(21) Appl. No.: 15/739,828

(22) PCT Filed: Jun. 27, 2016
(86) PCT No.: PCT/US16/39671

§ 371 Date: Dec. 26, 2017

Related U.S. Application Data
(60) Provisional application No. 62/185,180, filed on Jun. 26, 2015.
(60) Provisional application No. 62/185,915, filed on Jun. 29, 2015.

(51) Int. Cl. C12N 15/115; C12Q 1/6811

(52) U.S. Cl. CPC C12N 15/115; C12Q 1/6811; C12Q 2525/205; C12N 2330/31; C12N 2310/16

(57) ABSTRACT

Methods for preparing nucleic acid aptamers in the presence of a surfactant are described, as are methods for using nucleic acid aptamers to separate a target molecule from a sample. The methods may be used to separate abundant proteins including HSA from a biological sample such as serum.

### CROSS REFERENCE TO RELATED APPLICATIONS

This application claims the benefits of U.S. Provisional Patent Application Ser. No. 62/185,180, filed on Jun. 26, 2015, and U.S. Provisional Patent Application Ser. No. 62/185,915, filed on Jun. 29, 2015, which are hereby incorporated by reference in their entirety for all of their teachings.

### STATEMENT REGARDING FEDERALLY FUNDED RESEARCH

This invention was made with government support under Grant No. CHE 1308364 awarded by the National Science Foundation, Grant No. 59482CH awarded by the Army Research Office, and Grant Nos. 1R43GM108239-02 and 1R43CA203518-01 awarded by the National Institutes of Health. The government has certain rights in the invention.

### FIELD OF INVENTION

The disclosure provided herein relates to methods for preparing nucleic acid aptamers in the presence of a surfactant and optionally a reductant, and methods for using nucleic acid aptamers to separate a target molecule from a sample. For example, the methods disclosed herein may be used to separate abundant proteins from a biological sample such as serum or plasma.

### BACKGROUND

Serum and plasma are rich sources for the discovery of new peptide and protein biomarkers for disease diagnosis. However, these proteins are present in a relatively low abundance relative to proteins such as albumin. Thus, methods are needed to remove abundant serum proteins before methods such as mass spectrometry can be employed to detect low abundance biomarkers.

Nucleic acid aptamers can be used as affinity reagents to bind and remove abundant proteins (e.g. human serum albumin (HSA), IgG, fibrinogen) from human blood serum or plasma. Antibody-based columns are commercially available, but they can also remove low-abundance proteins that are bound to the high-abundance proteins. Carrying out protein depletion under denaturing and optionally also reducing conditions can reduce the pull-down of low abundance proteins. However, antibodies are not stable to these conditions. Nucleic acid aptamers can function in denaturing and reducing environments, and the selection of aptamers for human serum albumin (HSA) that will function under these conditions is disclosed herein. These aptamers may be immobilized on a solid phase and packed into a column to be used to deplete HSA from serum samples. For example, a series of aptamers can be developed for removal of the top 12 most abundant proteins from serum. Potential commercial products using aptamers may involve a variety of resins and pre-packed columns for use by proteomics researchers to deplete HSA and other abundant proteins from serum in order to facilitate biomarker discovery.

Proteomics is a powerful tool to assess the state of a cell, tissue or organism, including indications of disease. The technology disclosed herein provides novel methods to simplify complex protein mixtures derived from human fluids by removing abundant proteins that obfuscate the measurement of dilute potential protein biomarkers. It allows one to delve deeper into the proteome than existing state-of-the-art methods to discover new potential protein biomarker candidates that indicate disease, including cancers. This technology may be used as a research tool and to create diagnostic tests utilizing targeted proteomics methods. It also affects the discovery of diagnostic markers to identify and develop pharmaceuticals that are used to treat disease.

Nucleic acid aptamers have a number of advantages compared to antibodies, including greater ease of production and increased thermal stability. Aptamers are also capable of functioning in the presence of high concentrations of surfactants, which readily denature antibodies and other protein-based affinity reagents. Herein are disclosed experiments investigating the compatibility of nucleic acid aptamers with surfactants and optionally also with reductants. Neutral, zwitterionic and anionic surfactants have only a minor impact on the ability of aptamers to fold and bind hydrophilic target molecules. The compatibility of aptamers with commonly used surfactants expands their scope of potential applications, and the ability to modulate the substrate binding preferences of aptamers using a surfactant provides a novel route to increasing selectivity in analytical applications.

### SUMMARY

Novel methods to remove abundant proteins from complex biological fluid samples are disclosed, which can be used for developing a suite of methods to facilitate the de novo discovery of a complete set of low-abundance potential protein biomarkers. This technology can be used for the discovery of potential biomarkers for cancers.

The selective and specific removal of human serum albumin (HSA) and immunoglobulin G (IgG) isoforms and multimers and other abundant proteins from human plasma or serum is envisioned, as the albumins and IgG comprise about two thirds of the total protein in human serum or plasma, and cause dynamic range issues for both tandem mass spectrometry and difference gel electrophoresis (DIGE) approaches for biomarker discovery. The technology disclosed herein has the potential to remove up to three orders of magnitude of abundant proteins from serum or plasma, which is a 50-fold improvement over conventional state-of-the-art methods. The conditions are specifically chosen to minimize protein-protein interactions, which compromise the performance of existing methods. When this technology is combined with ever-improving LC/LC-MS/MS and high dynamic range DIGE imagers, the full dynamic range of the instrumentation can be explored, facilitating the search for new potential protein biomarkers.

The present disclosure provides methods for selecting a nucleic acid aptamer, the methods comprising the steps of providing a solution comprising a plurality of nucleic acids, a target molecule, a surfactant, and optionally a reducing agent whereupon at least one nucleic acid binds to the target molecule to form a complex, separating the complex from the solution, and separating the at least one nucleic acid from the complex, wherein the at least one nucleic acid is the nucleic acid aptamer.

The present disclosure also provides methods for separating a target molecule from a sample, the methods comprising contacting the sample with a nucleic acid aptamer selected against a target molecule, a surfactant and optionally a reducing agent, whereupon the nucleic acid aptamer forms a complex with the target molecule, and separating the complex from the sample.

Methods for purification of a biological sample are also disclosed herein, these methods comprising contacting the sample with a plurality of nucleic acid aptamers selected according to the method described above and a surfactant, whereupon at least one of the nucleic acid aptamers forms a complex with a target molecule, and separating the complex from the biological sample.

Other aspects of the present disclosure will become apparent by consideration of the detailed description and accompanying drawings.

### BRIEF DESCRIPTION OF THE DRAWINGS

The drawings below are supplied in order to facilitate understanding of the Description and Examples provided herein.

FIG. 1 is an image of an SDS-PAGE gel of the eluate desorbed from an Agilent Technologies® HSA-only MARS column. Lane 1 (furthest to the left) contains a MW ladder. Lane 2 contains untreated human plasma. Lanes 3 is blank. Lanes 4-10 contain, from left to right, progressively increasing amount of eluate ranging from 0.35 μg (lane 4) to 28 μg (lane 10).

FIG. 2 is a schematic illustration of an exemplary nucleic acid aptamer labeled with a fluorophore (F) which is annealed to a complementary strand labeled with a quencher (Q). In the presence of the aptamer target, the complementary strand is displaced and the target binding can be quantified by measurement with a fluorescence plate reader.

FIG. 3 is a graph of percent displacement of an exemplary aptamer by the aptamer target in the presence of buffer (open diamonds), a surfactant (solid diamonds), a reductant (squares), and both a surfactant and a reductant (circles).

FIG. 4 is a graph of the percent displacement of an exemplary aptamer by the aptamer target alone (buffer; diamonds) and in the presence of 1% of various surfactants, at increasing concentrations of the aptamer target. The surfactants are SDS (triangles), Triton X-100 (squares), Tween 20 (circles), CTAB (stars) and CHAPS (X's).

FIG. 5A is a graph showing the CD spectra for the DIS aptamer in the presence of buffer, 0.01% SDS, 1% SDS, 4% SDS, and 8 M urea.

FIG. 5B is a graph showing the CD spectra for the DCA aptamer in the presence of buffer, 0.01% SDS, 1% SDS, 4% SDS, and 8 M urea.

FIG. 5C is a graph showing the CD spectra for the BE aptamer in the presence of buffer, 0.01% SDS, 1% SDS, 4% SDS, and 8 M urea.

FIG. 6A is a graph of the percent displacement of an exemplary DOA aptamer by DOA (squares) or DIS (diamonds) with no surfactant present (i.e., in the presence of buffer).

FIG. 6B is a graph of the percent displacement of an exemplary DOA aptamer by DOA (squares) or DIS (diamonds) with in the presence of 0.01% SDS.

FIG. 6C is a graph of the percent displacement of an exemplary DOA aptamer by DOA (squares) or DIS (diamonds) with in the presence of 1% SDS.

FIG. 6D is a graph of the percent displacement of an exemplary DOA aptamer by DOA (squares) or DIS (diamonds) with in the presence of 4% SDS.

FIG. 7 is a schematic illustration of exemplary nucleic acid aptamers and aptamer targets when the surfactant SDS is added, which does not disrupt the aptamer binding but which can increase the selectivity of the aptamer due to preferential micelle encapsulation of certain targets.

FIG. 8A is a graph of the percent displacement of an exemplary DCA aptamer by DCA (squares) or DIS (diamonds) in buffer. The error bars represent the standard deviation of three independent trials.

FIG. 8B is a graph of the percent displacement of an exemplary DCA aptamer by DCA (squares) or DIS (diamonds) in the presence of 0.01% SDS. The error bars represent the standard deviation of three independent trials.

FIG. 8C is a graph of the percent displacement of an exemplary DCA aptamer by DCA (squares) or DIS (diamonds) in the presence of 1% SDS. The error bars represent the standard deviation of three independent trials.

FIG. 8D is a graph of the percent displacement of an exemplary DCA aptamer by DCA (squares) or DIS (diamonds) in the presence of 4% SDS. The error bars represent the standard deviation of three independent trials.

FIG. 9A is a graph of the percent displacement of an exemplary BE aptamer by BE (squares) or DIS (diamonds) in buffer. The error bars represent the standard deviation of three independent trials.

FIG. 9B is a graph of the percent displacement of an exemplary BE aptamer by BE (squares) or DIS (diamonds) in 0.01% SDS. The error bars represent the standard deviation of three independent trials.

FIG. 9C is a graph of the percent displacement of an exemplary BE aptamer by BE (squares) or DIS (diamonds) in 1% SDS. The error bars represent the standard deviation of three independent trials.

FIG. 9D is a graph of the percent displacement of an exemplary BE aptamer by BE (squares) or DIS (diamonds) 4% SDS. The error bars represent the standard deviation of three independent trials.

FIG. 10 is a bar chart showing the percent of initial DNA eluted in each round of SELEX in the first selection using the heat elution (red bars) or ligand elution (blue bars) protocol.

FIG. 11 is a bar chart showing the percent of initial DNA eluted in each round of SELEX in the second selection using the ligand elution protocol.

FIG. 12 is an image of an SDS-PAGE gel showing the eluate from an HSA gel shift assay.

FIG. 13 shows images of SDS-PAGE gels of a pull-down assay of HSA in the presence of SDS (upper image) or Tween 80 (lower image) and DTT, with (right lane) and without (left lane) an exemplary aptamer also present.

FIG. 14 is an image of an SDS-PAGE gel of a pull-down assay of HSA obtained from plasma (lane 1) or purified (lane 2), with (lane 4) and without (lane 3) an exemplary aptamer also present.

FIG. 15 is an image of an SDS-PAGE gel of a pull-down assay of HSA with no aptamer present (left lane), and with an aptamer present (right lane).

FIG. 16 is an image of an SDS-PAGE gel of a pull-down assay of HSA under various conditions; HSA alone (lane 1), with no aptamer present (lanes 2-3), and with an aptamer present (lanes 4-5).

### DETAILED DESCRIPTION

An effective subtractive method to remove abundant proteins from complex human fluid samples to facilitate de novo protein biomarker discovery is disclosed herein. This technology is motivated by a number of observations, including that existing state-of-the-art immunodepletion methods are simply not effective. A representative product, Agilent's MARS® column that targets the 14 most abundant serum proteins, removes only 95% of the protein mass from human plasma samples, which only reduces the protein concentration dynamic range by a factor of 20. This is not sufficient to reduce the ten order-of-magnitude plasma protein concentration dynamic range to the five order-of-magnitude concentration dynamic range of traditional proteomics methods, LC/LC-MS/MS and DIGE. Many interesting potential protein biomarkers, particularly for cancers, are thought to be present in only trace amounts, and hence cannot currently be detected.

Also, existing immunodepletion methods are not particularly selective: many potential protein biomarkers are bound to abundant proteins that are removed by immunodepletion, thus these bound proteins are removed too. Further, the largest columns currently available are inadequate for the large sample volumes that are required to obtain sufficient amounts of low abundance proteins for detection by proteomics methods. Finally, these columns are expensive: $29,000 for Agilent's column that removes 14 abundant proteins from 250 μL of plasma.

The performance of state-of-the-art immunodepletion columns is compromised by protein-protein associations owing to their separation of proteins in their native state. Blood proteins as a rule associate one with another; otherwise, the half of the proteins below the molecular weight cutoff of the kidney would be cleared quickly. Since de novo analytical methods do not require proteins to maintain their native conformation, high-performance and cost-effective adsorption media that use aptamers as recognition elements can be developed. To minimize protein-protein association, aptamers are selected with, and can be used under, denaturing conditions and optionally also reducing conditions, such as using solutions containing, for example, 4% sodium dodecyl sulfate (SDS) and 50 mM dithiothreitol (DTT). This SDS concentration is well above its critical micelle concentration, so hydrophobic small molecules, peptides and proteins may be retained within SDS micelles and pass through the column for detection, rather than adsorb non-selectively elsewhere.

The methods disclosed herein are useful for human plasma, but can also be used to target other protein sources, including different human biological fluids, fluids from animals, or other biological sources such as cell cultures or tissues.

Unlike antibodies, nucleic acid aptamers may be well-suited for targeting specific proteins in denatured and optionally also reduced human plasma samples. Aptamers are the nucleic acid equivalent of antibodies, as they have been generated to bind to specific native protein targets with high affinity and selectivity. While aptamers serve a similar function to antibodies, they possess two differences: (1) aptamers do not rely on disulfide bonds to maintain their structural integrity, and (2) aptamers are not affected by the dilute surfactants that are used ion selections. Further, in human serum end-functionalized aptamers are not degraded enzymatically and are able to bind target molecules.

For example, an aptamer set against HSA, IgG isoforms and other abundant proteins can be generated from a combinatorial library using the SELEX process performed under conditions including a surfactant, which can also optionally contain a reductant. Multicomponent targets and next-generation sequencing may also be used in the selection process. The active aptamers can be immobilized onto solid supports, such as porous or nonporous beads, capillary tubes, or walls of channels opened within a microfluidic device.

The aptamer adsorbents developed here potentially have a higher selectivity, specificity, and reproducibility, and are more cost effective and have longer lives than existing commercial products. Reducing the abundant protein mass by three or more orders of magnitude using the technology disclosed herein, combined with improved LC/LC-MS/MS methods and DIGE imager dynamic range improvements, can result in nearly the entire concentration dynamic range of proteins in plasma being measured. This may allow for the discovery of very dilute new protein biomarkers, targeting de novo protein biomarker discovery in general and cancer protein biomarker discovery in particular.

Proteins traditionally have been a rich source for disease and companion diagnostic tests, and for patient stratification. This is because many diseases are caused by changes in cellular networks resulting from genetic changes, environmental challenges, or both. Such perturbations can alter mRNA transcription, microRNA, and therefore the proteins a cell expresses. These protein expression alterations cause a change in the tissue-specific proteins, protein fragments or peptides shed into the tissue/organ microenvironment. Such altered protein expression profiles in human fluids emanating from the diseased tissue/organ constitute molecular signatures that reflect the original change in cell function.

Plasma is a convenient, minimally-invasive source for diagnostic tests. All proteins and peptides ultimately drain into the blood; the plasma proteome therefore is an overwhelmingly complex superset of an individual's proteome. There may be over 300,000 proteins in plasma when one accounts for glycosylation and other post-translational modifications, splice variants, and cleavage products. Also, the protein dynamic range in plasma spans more than 10 orders of magnitude, and there is no method akin to PCR available to amplify trace protein amounts.

Recently, the rate of new protein-based clinical diagnostic tests that have been introduced to the market has fallen substantially. This may be a consequence of an inadequate pool of potential protein or peptide biomarker candidates from which a suitable protein or peptide panel ultimately can be developed by targeted methods.

Rather than rely on a “preselected” and finite pool of recognition elements, such as is used by some commercial vendors, new sample pre-treatment methods that allow for the de novo discovery of new potential protein biomarkers are needed. Once pretreated, the existing proteomics toolbox, including one- and two-dimensional liquid chromatography-tandem mass spectrometry (LC- and LC/LC-MS/MS) and difference gel electrophoresis (DIGE) can be used to uncover an expanded and more complete potential protein biomarker pool. These methods have a dynamic range of only three to five orders of magnitude, so they cannot be used to determine potential biomarkers when abundant proteins are present. Both are far below the ten orders of magnitude required to detect trace proteins, peptides or small molecules in plasma where interesting potential new biomarkers may be found.

Delving deeper into the proteome of complex human fluids for new potential protein biomarkers clearly requires that the dynamic range of the plasma proteome be reduced. Most human fluids contain a disproportionately large amount of only a few proteins: the 12 common serum albumins contribute about 50% (or 40 mg/ml) of the plasma protein mass. The 22 most abundant proteins sum to 99% of the total protein mass. In contrast, low-abundance proteins such as prostate specific antigen and cytokines are present in the ng/ml and pg/ml range. These low-abundance proteins cannot be seen with de novo methods unless the high-abundance proteins are removed.

The identity of the abundant proteins is known, so immunodepletion methods using antibodies have been created that are intended to remove them specifically. These antibodies are bound to solid supports, and by passing a human fluid sample across them (via spin or HPLC formats), the abundant proteins can be retained while allowing those of lower abundance to pass through and be collected for analysis. Agilent® MARS columns are the best known of these, though there are many others. The antibody columns are then regenerated by displacing/eluting the retained abundant proteins, and the columns regenerated and reused (typically, 200 times).

Unfortunately, current immunodepletion methods are not sufficiently effective. They may simplify complex protein mixtures, but the additional proteome exposed after their use is underwhelming. The “top 14” abundant protein MARS column results in only a 95% reduction in total protein (according to the vendor), which is equivalent to a dynamic range compression of 20. Protein samples treated using MARS columns typically only increase the number of proteins identified by about 30% using either DIGE or LC-MS/MS approaches. This is a disappointingly small increase in light of the number of unique dilute proteins that are expected. Simply, the concentration dynamic range compression using existing immunodepletion methods is inadequate for de novo protein biomarker discovery.

An effective abundant protein removal method can have a number of attributes. First, the protein dynamic range can be compressed compared to the current state-of-the-art. A protein reduction of 99.9+%, or a dynamic range compression of over 1,000, is desirable, which would improve on the state-of-the-art by a factor of 50 or more. Second, the method can suppress protein-protein associations, so that dilute potential protein or peptide biomarkers are not co-removed with the abundant proteins. Existing methods remove abundant proteins without disturbing their native state, and proteins in blood naturally associate with others. Half the protein mass has a molecular weight below the 45 kDa cut-off of the kidney. If they did not associate with other larger proteins, these proteins would be cleared quickly from the blood and not be able to perform their useful functions.

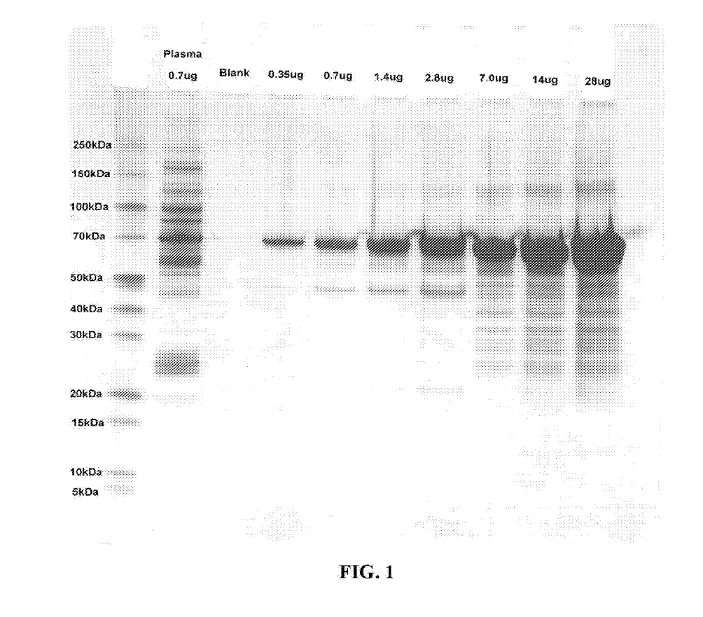

Removing abundant proteins under native conditions also removes other proteins. Shown in FIG. 1 is a representative SDS-PAGE image of the eluate protein desorbed from an HSA-only MARS column, with the eluate concentration increasing from 0.35 μg (left) to 28 μg (right). Lane 1 is a MW ladder, lane 2 is untreated human plasma, and lane 3 is blank. Lanes 4-10 contain increasing loadings of the protein retained on the column. These samples should contain only HSA (66 kDa) and its dimer (132 kDa) if there is no protein association; clearly numerous proteins other than albumin are present. Subsequent LC-MS/MS analysis showed that the protein diversity of the “HSA-only” fraction is 40% of the initial plasma; if HSA alone was removed, that diversity would be much less than 1%. There is a large overlap between these MS-identified proteins with those known to bind with HSA, indicating that retained proteins do not bind non-specifically with the support. Indeed, this binding of HSA to other proteins has led to the study of a subproteome known as the “albuminome,” where the change in binding of proteins to albumin is studied as a source of new protein biomarkers.

Finally, the abundant protein removal method is able to treat large sample volumes. To obtain enough protein to analyze from a sample, once three or more orders of magnitude of protein mass have been removed, initial plasma volumes on the order of milliliters or more are required. Fortunately, such large-volume samples are available from commercial vendors, who can supply ca. 20 mL plasma samples from an individual with diseased (such as lung cancer) and control samples drawn from the same cohort. The “top 14” MARS column, in addition to not being effective or selective, is also expensive when it is used to treat large sample volumes. The largest capacity column available treats a maximum of 250 μL of plasma per cycle. The column costs $29,000, and has a life of only 200 cycles, or a total of 50 ml of plasma. In addition, the column effectiveness changes with time: the columns that are purchased today are not the same as one from a month ago or a month in the future.

A method that removes 99.9+% of the more abundant proteins from human plasma is desirable. The methods disclosed herein are performed under denaturing and optionally also reducing conditions chosen to minimize protein-protein association, leaving the dilute potential protein biomarkers in solution. HSA is the most abundant protein in plasma. IgG is the second most abundant, and represents the immunoglobulins, the most diverse plasma protein family. When the inventive methods are combined with high-dynamic-range gel imagers and LC/LC-MS/MS instrumentation, nearly the entire plasma protein dynamic range may be covered and dilute potential protein biomarkers may be discoverable using the traditional (and ever improving) proteomics toolbox.

Disclosed herein are methods of using nucleic acid aptamers as recognition elements instead of antibodies against denatured and optionally also reduced abundant proteins. The inventive methods use background solution conditions that contain a surfactant, such as sodium dodecyl sulfate (SDS), and optionally also a reducing agent such as DTT or TCEP, which opens disulfide bonds. These conditions are similar to those used for SDS-PAGE, which is used exclusively over native PAGE to separate proteins unless one is specifically studying protein-protein association. For example, a native PAGE on 95% pure HSA (Gemini Bio Products) reveals 5 bands, one of which is the dimer. In contrast, SDS-PAGE on the same sample shows 14 bands, indicating a substantial protein disaggregation.

Prior to conducting the experiments disclosed herein, we could not predict whether denaturing and optionally reducing conditions would allow for the generation of selective aptamers or for binding of aptamers to target molecules. We did not know whether the structure of nucleic acid aptamers would be affected by surfactants and reducing agents. We also did not know if sufficient structure remained in the protein for aptamer binding in the presence of surfactant and optionally reductant. Surfactants such as SDS affect the secondary structure of proteins and reductants affect the tertiary and quaternary structure of a protein. Such protein structure changes result in protein disaggregation. Also, substantial surfactant binds to proteins (for SDS, on average about one surfactant molecule binds can bind to every protein or peptide amino acid). As such, we did not expect that we would be able to generate selective aptamers and/or effect binding between aptamers and target compounds under denaturing and optionally reducing conditions, because we expected the structure necessary to confer binding would be disrupted. In fact, denaturing conditions are generally used to release aptamers from target compounds, and other known affinity reagents (e.g., antibodies) generally do not bind to their targets under denaturing and/or reducing conditions. Disclosed herein is data showing that the target binding ability of nucleic acid aptamers is maintained under denaturing and optionally also reducing conditions, and that such aptamers are chemically stable and able to bind target molecules in human serum or plasma without detectable enzymatic degradation over several hours. We were surprised by this discovery that surfactants and reducing agents have little effect, if any, on aptamer binding to a target compound.

Aptamers have certain practical advantages as compared to antibodies, in addition to their ability to function under conditions that disrupt protein-protein associations. The large size and complex structure of antibodies dictates that they must be produced either in live animals or cell cultures, which leads to high cost and problematic batch-to-batch variation. In comparison, aptamers have a relatively smaller size and can be folded properly in vitro, enabling functional aptamers to be obtained via low-cost chemical oligonucleotide synthesis. This reduces the cost and time required for production, and increases reliability, as different batches of aptamers (once purified to remove truncated byproducts) are chemically identical to one another. Moreover, for the protein depletion applications disclosed herein, strongly denaturing conditions can be employed to elute bound proteins efficiently, then the aptamers re-fold for subsequent use. The ability of nucleic acids to withstand repeated cycles of denaturation and re-folding translate into longer lifetimes, and thus better value, for the abundant protein depletion columns contemplated here.

One feature of this technology is carrying out nucleic acid aptamer selections for denatured and optionally also reduced protein targets, as the vast majority of reported aptamers bind to folded proteins. To obtain aptamers capable of binding to denatured and optionally also reduced proteins, the selections are carried out in the presence of a surfactant and optionally also a reductant, such as 4% SDS and 50 mM DTT. A second feature is that the selections can be multiplexed, that is, aptamer selections are against multiple isoforms, and the analytical methods then are used to choose the proper aptamer complement.

Nucleic acid aptamers hold significant promise for replacing antibodies in analytical applications, as aptamers are capable of binding to a wide variety of small-molecule, peptide and protein targets. The most commonly cited benefits of aptamers relative to antibodies include their ability to retain function after thermal denaturation, and the fact that they are chemically synthesized, which reduces both cost and batch-to-batch variation. Antibodies and other proteins are readily denatured by surfactants, as the hydrophobic portion of the surfactant can interact with hydrophobic surfaces on the protein, reducing the enthalpic cost of protein unfolding in aqueous medium.

The surfactants also provide a unique dimension of control over the substrate binding preferences of aptamers. At low concentrations, amphiphilic surfactant molecules are dispersed in solution and form a monolayer at the air-water interface. However, at concentrations above the critical micelle concentration (CMC) of the surfactant, self-assembly occurs to form micelles. These spherical or ellipsoidal structures possess a hydrophobic core that is capable of sequestering nonpolar molecules. As a result, surfactants are commonly used for applications such as purification and reaction catalysis. In the context of aptamer-target binding, analytes show variable partitioning into the micelle core depending upon their hydrophobicity, effectively increasing the selectivity of aptamers towards hydrophilic analytes. Substrate binding selectivity is important in many applications of aptamers, and previous studies have explored approaches to modulating selectivity through sequence mutation, incorporation of unnatural bases, or the addition of hydrophobic groups near the binding pocket of the aptamer. Due to the nature of these chemical modifications, they typically increase binding affinity for hydrophobic targets. Thus, the use of surfactants offers a complementary approach to modulating the substrate binding selectivity of aptamers.

Here, three general applications of this technology are disclosed: (1) generating a set of nucleic acid aptamers for HSA and IgG isoforms representative of those present in human plasma under denaturing and optionally also reducing solution conditions, (2) developing solid support immobilization methods and characterizing the adsorption and desorption behavior of reference compounds, and (3) demonstrating the effectiveness of the immobilized aptamer adsorbents by removing abundant proteins from denatured and optionally also reduced human plasma.

Aptamer Selection and Performance under Denaturing and optionally also Reducing Conditions. To explore the ability of aptamers to maintain their substrate-binding capability under the conditions required to disrupt protein-protein associations, the performance of an aptamer biosensor in the presence and absence of a surfactant, SDS, and a reducing agent, DTT, was compared. Although targeted for the generation and utilization of aptamers for a protein, a protein-binding aptamer could not be used for the initial testing, as those aptamers have been selected to bind to folded proteins. Thus, even if the aptamer retained its structure and function, the target may be compromised in such a way as to preclude binding.

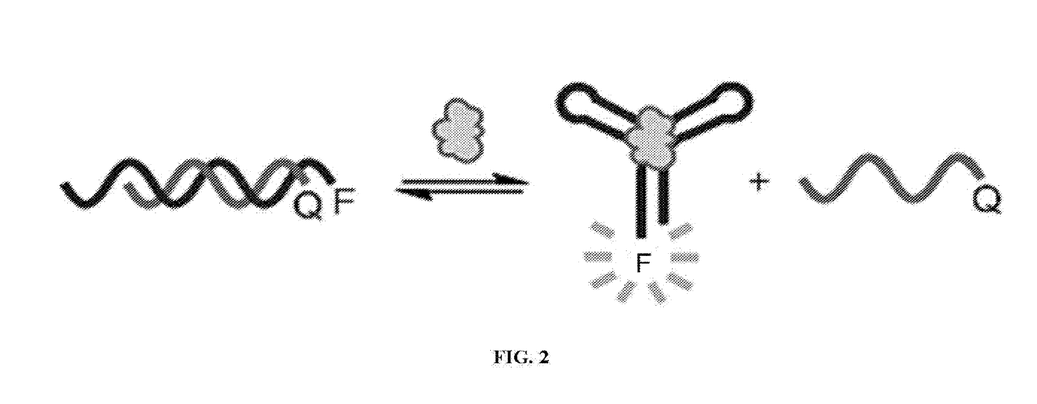

Instead, testing was carried out using the DNA aptamer for the small-molecule steroid, dehydroiso-androsterone-3-sulfate sodium salt (DIS). FIG. 2 is a schematic illustration of a fluorescent biosensor with a nucleic acid aptamer labeled with a fluorophore (F) which is annealed to a complementary strand labeled with a quencher (Q). As shown in FIG. 2, the short complementary strand is displaced in the presence of the DIS target. The aptamer and complementary strand are labeled with a fluorophore and a quencher, respectively, enabling quantification of target binding using a fluorescence plate reader.

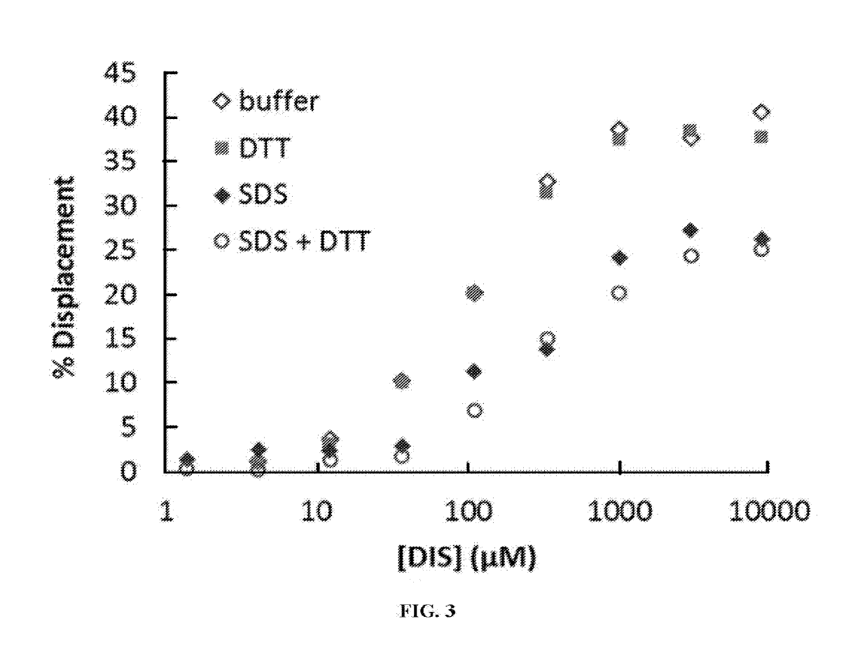

FIG. 3 is a graph of percent displacement of an exemplary nucleic acid aptamer by the aptamer target in the presence of buffer (open diamonds), 4% SDS surfactant (solid diamonds), 50 mM of DTT reductant (solid squares), or both 4% SDS and 50 mM DTT (open circles). This graph shows that target binding occurs in the presence of 4% SDS, alone and with 50 mM DTT.

The data in FIG. 3 show that the fluorescence response follows a nearly identical trend in the presence and absence of 4% SDS and 50 mM DTT. The slightly lower signal gain in the presence of SDS is likely a result of fluorophore quenching, as the binding curves still indicate similar Kd values. Importantly, this fluorescent biosensor format was employed only to demonstrate the ability of aptamers to function under denaturing and optionally also reducing conditions, and thus issues of fluorescence quenching will not be problematic in other applications. Also, it is noted that the small-molecule-binding aptamer employed for these studies has a mid μM Kd value, whereas protein-binding aptamers typically have Kd values in the mid nM range.

Aptamer Immobilization on Solid Supports. The nucleic acid aptamer can be linked to a solid support that has negligible non-specific adsorption of other plasma proteins while remaining active against its target. Solid supports include porous and nonporous beads, the interior wall of capillary tubes, and the walls of openings within microfluidic devices. Materials can be drawn from glasses (silica, fused silica and quartz), polymers such as poly(methyl methacrylate) polydimethylsiloxane and cyclic olefin copolymer, and agarose and related materials such as sepharose. The surface of these materials must be reacted with compounds that present to solution a reactive group to which the aptamer can be coupled. Such surface reactions are known to those skilled in the art.

One example for silica is a derivatization with trimethoxyglycidoxysilane under acidic conditions and oxidization from the diol to the aldehyde with periodate (reacts with primary amines and some secondary amines). Agarose can be derivatized with NHS. An aptamer terminated with a primary amine can be used, as it can be linked with a silica surface, the NHS agarose, and any linkers (with EDC). Unreacted sites can be passivated with ethanolamine or short-chain PEGs.

The process can work as follows. A slug of denatured and optionally also reduced plasma is injected into a column containing the immobilized aptamer, and the pass-through is collected. Once the aptamer is saturated and plasma breaks through the column, it is washed to remove interstitial fluids. The protein then is desorbed with urea, followed by re-equilibration in a surfactant and optionally also a reductant. Exemplary regeneration solutions include PBS containing increasing urea amounts from 1 M to 8 M, alone and with 25 mM DTT. High concentrations of urea can denature aptamers to release protein and promote the solubility of the proteins removed from the aptamer, and if low concentrations are not fully effective, thiourea can be added or guanidine can be used.

The aptamers generated against one or more target molecules can be used to determine an adsorpotion isotherm in the presence of surfactant and optionally reductant. These data can then be used to size an appropriate adsorption column. Continuous target samples can be injected with increasing concentration to confirm the isotherm using, for example, frontal analysis chromatography. The beds can be washed, and the desorption solutions evaluated. A flow rate analysis can be performed to determine proper flow rates in the presence of dispersion, again with frontal analysis chromatography by using the equation moments to obtain the appropriate parameters.

Finally, plasma samples can be tested on the HSA aptamers, under denaturing and optionally also reducing conditions. These results can be compared with the HSA-only MARS column. The proteins recovered from column desorption can be determined by SDS-PAGE, DIGE or LC-MS/MS.

Aptamer bed composition. The top HSA aptamers and/or other abundant protein aptamers can then be synthesized. HSA aptamers can be screened using many techniques to those practiced in the art, including free solution electrophoresis (FSE), to reject aptamers that bind poorly. Preliminary experiments with 2D gels show that the pure HSA contains isoforms that are very similar to plasma. Multiple components can be differentiated by using different initial aptamer concentrations; multiple rounds may be necessary if binding for different aptamers is comparable. It may be that the isoforms vary enough structurally that multiple aptamers will be required for the complete removal of all the isoforms.

The highest-binding aptamer can be immobilized, packed into a column and tested by injecting continuously the 95% pure HSA. If appreciable albumin is present in the pass-through before breakthrough, additional aptamers may be required. Further rounds may not be necessary once there is no appreciable HSA in the effluent before breakthrough. If multiple aptamers are necessary, the HSA isoform can be identified with 2D gels or LC-MS/MS methods.

Demonstration of Adsorbent on Human Plasma. The aptamer bed can then be tested against plasma, since its albumin composition may vary slightly from a pure sample. Changes in bed composition may be required, and can be generated as above, but using plasma as the starting material. A similar method can then be applied to other abundant proteins. If immobilized aptamers perform differently than the FSE results, additional screening rounds or sequence engineering may be required, or all of the aptamers can be immobilized and tested individually. The composition of the column retentate can be measured as the beds are developed. The aptamer-bound protein can be desorbed and analyzed initially with 2D gels, and with LC-MS/MS as necessary.

The performance of the denatured and optionally also reduced HSA/IgG aptamer bed can be compared with commercially available columns and depletion kits. The removal efficiency (the HSA/IgG in the pass-through before break through) and the retentate protein diversity, defined as the number of unique proteins identified by label-free LC-MS/MS in the column pass-through divided by the number of proteins identified in the initial plasma, can be compared.

Scale-up and cycling. A mixed aptamer bed that removes HSA and other abundant proteins can be scaled to accommodate a 250 μL plasma volume, which is the largest volume Agilent's “top 14” column can accept or larger plasma volumes. The bed can be subjected to repeated cycles of plasma injection, and the pass through, wash, and desorption eluent collected and analyzed. The inventive aptamer adsorbent developed here will likely show improved selectivity (see FIG. 1) and less degradation with time than the commercial product.

Using this technology, a >>95% depletion of HSA and IgG from human plasma can potentially be achieved, with a protein diversity of <1%, as compared to 40% for the commercial product. Material balances show that a suitable target for the HSA and IgG Kd values is on the order of 40 μM; the targeted Kd values are less than 10 μM. Further, a 250 μL plasma sample can potentially be analyzed, comparable to the largest available commercial product, to the above specification in less than four hours.

The methods disclosed herein include a method for selecting a nucleic acid aptamer, the method comprising providing a solution comprising a plurality of nucleic acids, a target molecule and a surfactant, whereupon at least one nucleic acid binds to the target molecule to form a complex, separating the complex from the solution, and separating the at least one nucleic acid from the complex, wherein the at least one nucleic acid is the nucleic acid aptamer.

In certain embodiments, the solution further comprises a reductant. In an embodiment, the reductant is DTT, TCEP or a combination thereof. The method may additionally include a step of heating the solution. In some embodiments, the target molecule comprises a small molecule, a peptide or a protein. For example, the target molecule comprises at least one of HSA, an immunoglobulin, or a steroid.

In some embodiments, the surfactant comprises at least one of a non-ionic, zwitterionic or anionic surfactant. For example, the surfactant may comprise at least one of SDS, Triton X-100 (i.e., polyethylene glycol p-(1,1,3,3-tetramethylbutyl)-phenyl ether), Tween 80 (i.e., polyethylene glycol p-(1,1,3,3-tetramethylbutyl)-phenyl ether), Tween 20 (i.e., polyoxyethylene (20) sorbitan monolaurate), Empigen BB (i.e., N,N-Dimethyl-N-dodecylglycine betaine), or CHAPS (i.e., 3-[(3-Cholamidopropyl)dimethylammonio]-1-propanesulfonate), among others. The surfactant may be present in an amount of at least 0.1% (w/v) of the solution, at least 0.2% (w/v) of the solution, at least about 0.3% of the solution, at least about 0.4% of the solution, at least about 0.5% of the solution, at least about 0.6% of the solution, at least about 0.7% of the solution, at least about 0.8% of the solution, at least about 0.9% of the solution, at least 1% (w/v) of the solution, at least 2% (w/v) of the solution, at least 3% (w/v) of the solution, or at least 4% (w/v) of the solution. In some embodiments, the nucleic acid aptamer may comprise RNA, DNA or any combination thereof. The nucleic acid aptamer also may comprise nucleic acids that include one or more non-naturally occurring nucleotides, such as, for example.

In some embodiments, the method for selecting a nucleic acid aptamer may comprise a nucleic acid aptamer comprising RNA or DNA, a target molecule comprising HSA, a surfactant comprising SDS or Tween, and a reductant comprising DTT or TCEP.

The methods disclosed herein also include a method for separating a target molecule from a sample, the method comprising contacting the sample with a nucleic acid aptamer selected according to the method described above and a surfactant, whereupon the nucleic acid aptamer forms a complex with the target molecule, and separating the complex from the sample. In certain embodiments, the surfactant is the same surfactant used in the selection of the nucleic acid aptamer according to the method described above.

In some embodiments, the solution further comprises a reductant. For example, the reductant may be DTT, TCEP or a combination thereof. In some embodiments, the target molecule comprises a small molecule or protein. For example, the target molecule comprises at least one of HSA, an immunoglobulin, or a steroid.

In further embodiments, the surfactant comprises at least one of a non-ionic or anionic surfactant. For example, the surfactant comprises at least one of SDS, Triton X-100 or Tween. The surfactant may be present in an amount of at least 0.1% (w/v) of the solution, at least 1% (w/v) of the solution, or at least 4% (w/v) of the solution. In an embodiment, the nucleic acid aptamer comprises RNA, DNA or any combination thereof.

In an embodiment, the nucleic acid aptamer used in the method for separating a target molecule from a sample, is attached to a solid support. For example, the solid support may be a bead, silica gel, CPG, quartz, fused silica, a polymer, or any combination thereof. The nucleic acid aptamer may be attached to the solid support via an amide linkage, a NHS linkage, a thiol linkage, a maleimide linkage, an azide linkage, an epoxide linkage, or any combination thereof.

In further embodiments, the method for separating a target molecule from a sample additionally comprises separating the target molecule from the complex. In certain embodiments, the method for separating a target molecule from a sample comprises providing a solution comprising a plurality of nucleic acids, a target molecule and a surfactant, whereupon at least one nucleic acid binds to the target molecule to form a complex, separating the complex from the solution, and separating the at least one nucleic acid from the complex, wherein the at least one nucleic acid is the nucleic acid aptamer, and wherein the nucleic acid aptamer comprises RNA or DNA, the target molecule comprises HSA, the surfactant comprises SDS or Tween, and the reductant comprises DTT or TCEP.

The methods disclosed herein further include a method for the purification of a biological sample, the method comprising contacting the sample with a plurality of nucleic acid aptamers selected according to the method described above and a surfactant, whereupon at least one of the nucleic acid aptamers forms a complex with a target molecule, thereby allowing for separation of the target molecule from the sample.

In certain embodiments, the surfactant is the same surfactant used in the selection of the nucleic acid aptamer according to the method described above.

In some embodiments, the solution further comprises a reductant. For example, the reductant may be DTT, TCEP or a combination thereof. In some embodiments, the target molecule comprises a small molecule or protein. For example, the target molecule comprises at least one of HSA, an immunoglobulin, or a steroid.

In further embodiments, the surfactant comprises at least one of a non-ionic or anionic surfactant. For example, the surfactant comprises at least one of SDS, Triton X-100 or Tween. The surfactant may be present in an amount of at least 0.1% (w/v) of the solution, at least 1% (w/v) of the solution, or at least 4% (w/v) of the solution. In an embodiment, the nucleic acid aptamer comprises RNA, DNA or any combination thereof.

In an embodiment, the nucleic acid aptamer used in the method for separating a target molecule from a sample, is attached to a solid support. For example, the solid support may be a bead, silica gel, CPG, quartz, fused silica, a polymer, or any combination thereof. The nucleic acid aptamer may be attached to the solid support via an amide linkage, a NHS linkage, a thiol linkage, a maleimide linkage, an azide linkage, an epoxide linkage, or any combination thereof. In certain embodiments, the solid support comprises a mixed bed or cartridge column.

In some embodiments, the biological sample is at least one of blood, plasma, serum, cerebrospinal fluid (CSF), pleural effusion fluid, saliva, tears, urine, a cell lysate or a tissue extract. The method may further comprise separating the target molecule from the complex.

In further embodiments, the method for the purification of a biological sample comprises contacting the sample with a plurality of nucleic acid aptamers selected according to the method described above and a surfactant, whereupon at least one of the nucleic acid aptamers forms a complex with a target molecule, and separating the complex from the biological sample, wherein the biological sample comprises human plasma, the nucleic acid aptamer comprises RNA or DNA, the target molecule comprises HSA, the surfactant comprises SDS, Tween, or Empigen, and the reductant comprises DTT or TCEP.

In some embodiments, the methods disclosed herein include a method for the selection of DNA aptamers in the presence of one or more nonionic, anionic, or amphoteric surfactants, one or more reducing agents, or both a surfactant and a reducing agent, to generate aptamers having affinity to target species, including small molecules (such as steroids or drugs of abuse) or proteins. In certain embodiments, the selection can be performed wherein the surfactant is selected from at least one of Triton X-100, Tween, sodium dodecyl sulfate (SDS), or Empigen.

In an embodiment, the methods disclosed herein include a method for the use of one or more DNA aptamers in the presence of aqueous solutions of one or more nonionic, anionic or amphoteric surfactants, one or more reducing agent, or both a surfactant and a reducing agent, to bind to one or more target species, including but not limited to, small molecules or proteins, to remove one or more of those target species from human or animal fluid samples, such as (but not limited to) blood, plasma, serum, cerebrospinal fluid, pleural effusion fluid, saliva, tears, or urine, and from cell lysates or tissue extracts. In further embodiments, the protein is selected from at least one of human serum albumin, an immunoglobulin (IgG, IgA, IgM, IgD, IgE), antitrypsin, transferrin, fibrinogen, haptoglobulin, alpha 2 macroglobulin, alpha 1 acid glycoprotein, apolipoprotein A1 and A2, complement C3 or transthyretin.

The methods described herein include those wherein the DNA aptamer is chemically linked to a solid material including, but not limited to, porous and nonporous beads, the internal walls of capillary tubes, and the surface of openings within microfluidic devices. Materials include silicas (silica gel, controlled-pore glass, quartz, fused silica) and polymers (such as polystyrene, poly(methylmethacrylate), cyclic olefin copolymer, and polycarbonate) and agarose and related materials such as sepharose. In certain embodiments, the chemical linkage is selected from at least one of an amine-carboxylic acid linkage, an amine-NHS ester linkage, a thiol-maleimide linkage, an azide-alkyne linkage, or an amine-epoxide linkage.

The methods described herein also include those wherein the surfactant modifies the substrate binding preference of a DNA aptamer by sequestering hydrophobic ligands within surfactant micelles. In some embodiments, the surfactant is selected from at least one of Triton X-100, Tween 20 or 80, sodium dodecyl sulfate (SDS), or Empigen BB.

Before any embodiments of the invention are explained in further detail, it is to be understood that the invention is not limited in its application to the details of construction and the arrangement of components set forth in the following description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof, as well as additional items.

It also should be understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

It should be understood that, as used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 10%. In all cases, where the term “about” has been used to describe a value, it should be appreciated that this disclosure also supports the exact value.

Reference throughout this specification to “one embodiment,” “an embodiment,” or similar language means that a particular feature, structure, or characteristic described in connection with the embodiment is included in at least one embodiment of the invention provided herein. Thus, appearances of the phrases “in one embodiment,” “in an embodiment,” and similar language throughout this specification may, but do not necessarily, all refer to the same embodiment.

Furthermore, the described features, structures, or characteristics of the methods provided herein may be combined in any suitable manner in one or more embodiments. In the following description, numerous specific details are provided, to provide a thorough understanding of embodiments. One skilled in the relevant art will recognize, however, that the embodiments may be practiced without one or more of the specific details, or with other methods, components, materials, and so forth. In other instances, well-known structures, materials, or operations are not shown or described in detail to avoid obscuring aspects of the embodiments.

### EXAMPLES

Exemplary embodiments of the present disclosure are provided in the following examples. The examples are presented to illustrate the inventions disclosed herein and to assist one of ordinary skill in making and using the same. These are examples and not intended in any way to otherwise limit the scope of the inventions disclosed herein.

EXAMPLE 1.

To explore the effect of surfactants on aptamer function and substrate binding preference, a series of structure-switching DNA aptamer biosensors previously reported that bind to steroid targets were used. These steroid targets are shown below in Scheme 1.

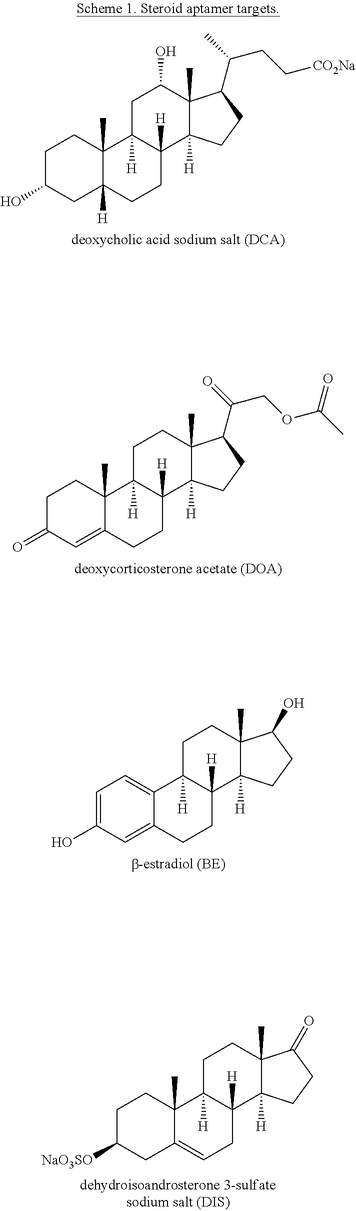

Each structure-switching biosensor was comprised of an aptamer and a short complementary strand, which are functionalized with a fluorophore and quencher, respectively, as shown in FIG. 2. In the absence of the target molecule, the complementary strand binds to the aptamer and fluorescence is quenched. However, in the presence of a target that binds to the aptamer, the complementary strand is displaced, resulting in a dose-dependent increase in fluorescence signal.

Here, it is shown that the aptamers maintain their secondary structure and substrate binding capability in the presence of neutral and anionic surfactants, and that the presence of surfactant can be used to modulate substrate binding preference to favor more hydrophilic ligands. The ability of aptamers to function in the presence of surfactants expands their scope of potential applications. Additionally, the ability to modulate the substrate binding preferences of aptamers using a simple additive provides a novel route to increasing selectivity in analytical applications.

General. All DNA was purchased from the University of Utah DNA/Peptide Synthesis Core Facility, where it was synthesized using phosphoramidites and CPG cartridges from Glen Research. All other materials were purchased from commercial suppliers and used without further purification. Absorbance and fluorescence measurements were recorded using a Biotek Synergy Mx microplate reader.

| | | Catalog | Purity/ |
|---|---|---|---|
| Chemical | Supplier | Number | Composition |
| Triton X-100 | CalBioChem | 648468 | — |
| Tween 20 | Sigma Life Sciences | P1379 | 47% Lauric |
| | | | acid |
| Sodium dodecyl sulfate (SDS) | Research Products | L22010 | ≥99% |
| | International Group | | |
| Cetyl trimethylammonium bromide (CTAB) | Sigma Aldrich | H5882 | ≥98% |
| 3-[(3-cholamidopropyl)dimethylammonio]- | Sigma Aldrich | C3023 | 98% |
| 1-propanesulfonate (CHAPS) | | | |
| Dehydroisoandrosterone 3-sulfate | Sigma Aldrich | D5297 | ≥98% |
| sodium salt dihydrate (DIS) | | | |
| Deoxycholic acid sodium salt (DCA) | Sigma Aldrich | 30170 | ≥98% |
| Beta-Estradiol (BE) | Sigma Aldrich | E8875 | ≥98% |
| Deoxycorticosterone acetate (DOA) | Sigma Aldrich | D7000 | 97.8% |

Preparation of stock solutions. All samples were prepared in a buffer containing 20 mM Tris, 150 mM NaCl, pH 7.4. The aptamer and complementary strand were annealed by incubating at 90° C. for 5 min followed by rapid cooling. The following DNA concentrations were used for each biosensor: DIS, 1 μM aptamer and 2 μM displacement strand; BE, 0.15 μM aptamer and 0.30 μM displacement strand; DCA, 1 μM aptamer and 2 μM displacement strand.

Stock solutions of surfactants were prepared by dissolving sodium dodecyl sulfate (SDS), cetyl trimethylammonium bromide (CTAB), 3-[(3-cholamidopropyl)dimethylammonio]-1-propanesulfonate (CHAPS), Triton X-100, or Tween 20 in Tris buffer at 5 or 10% (w/v). Ligand solutions were prepared by dissolving each steroid in DMSO (DCA, BE, DIS) or 2:1 CHCl₃:DMSO (DOA) at 500 mM then performing a 3-fold dilution series in DMSO to maintain the concentration of organic solvent in all samples constant at 2%.

Fluorescence measurements. For initial testing of surfactant scope, solutions were prepared having 0% or 1% (w/v) surfactant. For monitoring the effects of increasing SDS, solutions were prepared having 0, 0.01, 1, or 4% SDS. The DNA stock solution and surfactant were combined in Tris buffer and allowed to equilibrate for 5 min. The ligand was then added, and the solutions were incubated for 20 min at 25° C. Fluorescence measurements were then acquired with λex=495 nm and λem=525 nm at 25.0±0.2° C. The percent displacement (% D) for each biosensor was calculated using Equation 1:

$$\% D = ( \frac{F - {F}_{0}}{{F}_{m} - {F}_{0}} ) * 100 , \qquad Equation ( 1 )$$

where F is the measured fluorescence, F₀ is the fluorescence of the biosensor in the absence of ligand, and Fₘ is the fluorescence of the aptamer alone.

Circular dichroism (CD) analysis. CD spectra were acquired using a JASCO J815 CD spectrometer. The CD spectra were collected using unlabeled aptamers (10 μM) prepared in Tris buffer containing 0, 0.01, 1, or 4% SDS. As a positive control for denaturation, CD spectra were acquired for each aptamer in Tris buffer with 8 M urea. Following heating and cooling, the aptamer strands were incubated at 25° C. for 2 hours. All CD spectra were recorded at 23° C. scanning from 220 to 320 nm at 100 nm/min (cell path length=2.00 mm). Final spectra are an average of 6 scans.

Choice of aptamer sequences. To investigate the effect of surfactants on aptamer-ligand recognition, it was necessary to use aptamers that bind to small-molecule, rather than protein, targets. This is because nearly all protein-binding aptamers have been selected to recognize folded proteins, and thus even if the aptamer retained its structure and function, the addition of surfactant would compromise the protein target in such a way as to preclude binding. Aptamers that had been reported in a structure-switching biosensor format were utilized, as this enabled convenient fluorescence-based monitoring of target binding. Thus, three aptamer biosensors previously reported that bind to small-molecule steroid targets were used.

TABLE 1
Sequences of DNA aptamers (A)
and complementary strands (CS).

| SEQ | | |
|---|---|---|
| ID NO | Name | Sequence (5′→3′) |
| 1 | BE-A | FAM-CTC TCG GGA CGA CAT GGA TTT TCC |
| | | ATC AAC GAA GTG CGT CCG TCC CG |
| 2 | BE-CS | GTC GTC CCG AGA G-BHQ1 |
| 3 | DCA-A | FAM-CTC TCT CGG GAC GCT GGG TTT TCC |
| | | CAG GAC GAA GTC CGT CCC GA |
| 4 | DCA-CS | CGT CCC GAG AGA-BHQ1 |
| 5 | DIS-A | FAM-CTG CTC TCG GGA CGT GGA TTT TCC |
| | | GCA TAC GAA GTT GTC CCG AG |
| 6 | DIS-CS | GTC CCG AGA GCA-BHQ1 |

FAM = fluorescein;
BHQ1 = Black Hole quencher 1.

These aptamers were selected using the steroid targets DCA, DIS, and BE (as shown in Scheme 1), and were intentionally selected to have a broad substrate scope, with each aptamer sequence having affinity towards multiple steroid targets.

Exploring the effect of surfactant type. The DIS aptamer was chosen as a model to survey the effect of varying surfactant types on substrate binding. Using five common surfactants that represent all four ionic states (i.e. cationic, anionic, nonionic, and zwitterionic), the fluorescence response of the aptamer biosensor to DIS in the presence of 1% (w/v) of each surfactant was measured. The 1% concentration used in these studies is above the CMC for each of the surfactants, ensuring the formation of micelles.

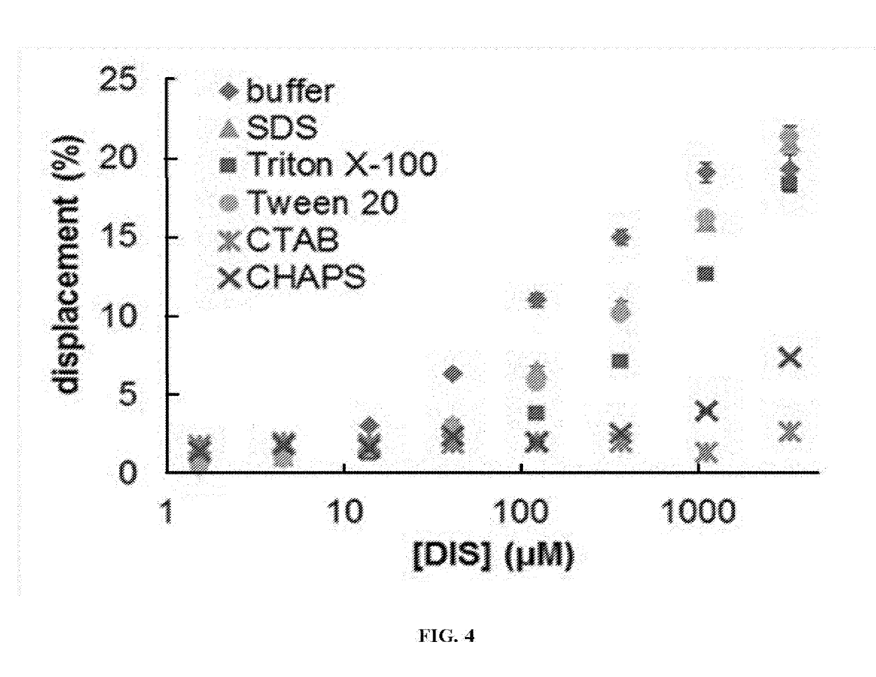

FIG. 4 shows the response of the DIS biosensor to increasing concentrations of DIS ligand alone (buffer; diamonds) and in the presence of 1% of the commonly used surfactants SDS (triangles), Triton X-100 (squares), Tween 20 (circles), CTAB (stars) and CHAPS (x's). The error bars represent the standard deviation of three independent trials.

The data in tabular form for the average percent displacement (% dis.) and standard deviation (s.d.) for three trials using the DIS biosensor in the presence of various surfactants is shown below in Table 2:

TABLE 2
Displacement data for DIS alone (buffer) and with 1% surfactant.

| [DIS] | buffer | | SDS | | CTAB | |
|---|---|---|---|---|---|---|
| (μM) | % dis. | s.d. | % dis. | s.d. | % dis. | s.d. |
| 3333 | 19.29 | 0.92 | 21.02 | 0.44 | 2.64 | 0.24 |
| 1111 | 19.06 | 0.58 | 15.87 | 0.39 | 1.33 | 0.07 |
| 370 | 15.00 | 0.47 | 10.65 | 0.26 | 1.96 | 0.10 |
| 123 | 11.01 | 0.41 | 6.62 | 0.19 | 2.03 | 0.10 |
| 41.2 | 6.28 | 0.19 | 3.16 | 0.23 | 1.91 | 0.07 |
| 13.7 | 3.01 | 0.09 | 2.06 | 0.05 | 1.89 | 0.08 |
| 4.57 | 1.39 | 0.05 | 1.06 | 0.03 | 1.97 | 0.06 |
| 1.52 | 0.72 | 0.03 | 1.49 | 0.05 | 1.76 | 0.07 |
| [DIS] | CHAPS | | Tween 20 | | Triton X-100 | |
| (μM) | % dis. | s.d. | % dis. | s.d. | % dis. | s.d. |
| 3333 | 7.32 | 0.13 | 21.32 | 0.72 | 18.31 | 0.44 |
| 1111 | 3.93 | 0.09 | 16.18 | 0.42 | 12.61 | 0.45 |
| 370 | 2.56 | 0.06 | 10.12 | 0.18 | 7.07 | 0.03 |
| 123 | 2.02 | 0.06 | 5.78 | 0.12 | 3.78 | 0.07 |
| 41.2 | 2.35 | 0.04 | 2.94 | 0.08 | 1.93 | 0.06 |
| 13.7 | 1.64 | 0.03 | 1.48 | 0.05 | 1.25 | 0.04 |
| 4.57 | 1.81 | 0.05 | 0.82 | 0.03 | 0.89 | 0.03 |
| 1.52 | 1.47 | 0.05 | 0.40 | 0.01 | 0.85 | 0.03 |

In the presence of SDS, Tween 20, or Triton X-100, the biosensor showed only a slightly attenuated response compared to its behavior in pure buffer (see FIG. 4). However, the biosensor shows no detectible response in the presence of positively charged CTAB, and in zwitterionic CHAPS, the biosensor only begins to show response at the highest DIS concentrations. These results are consistent with the hypothesis that surfactants having a positively charged functional group are more likely to interact with the negatively charged DNA backbone. In fact, a 2% CTAB solution is often used for DNA precipitation. SDS was selected as the surfactant used for all further studies, as the biosensor performed well in this surfactant, and SDS is frequently used for protein denaturation.

Structural analysis using CD spectroscopy. The ability of the DIS aptamer to bind its target molecule in the presence of 1% SDS suggests that this concentration of surfactant does not significantly disrupt DNA folding. To validate this idea and explore the tolerance of DNA folding to increased concentrations of SDS, CD spectra were acquired for each of the three aptamers in the presence of 0, 0.01, 1, and 4% SDS. These SDS concentrations were chosen as they allow comparison of DNA secondary structure at SDS concentrations below (0 and 0.01%) and above (1 and 4%) the CMC. As a positive control to ensure that a change in the CD spectrum would be observed upon DNA unfolding, spectra for each aptamer in the presence of 8 M urea were acquired, which is a concentration known to denature DNA secondary structure.

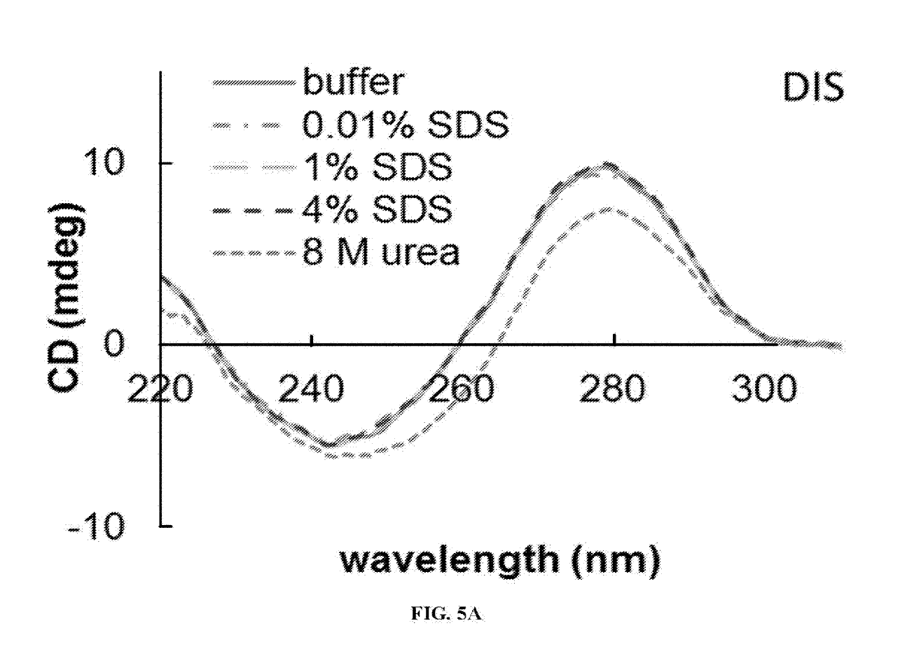

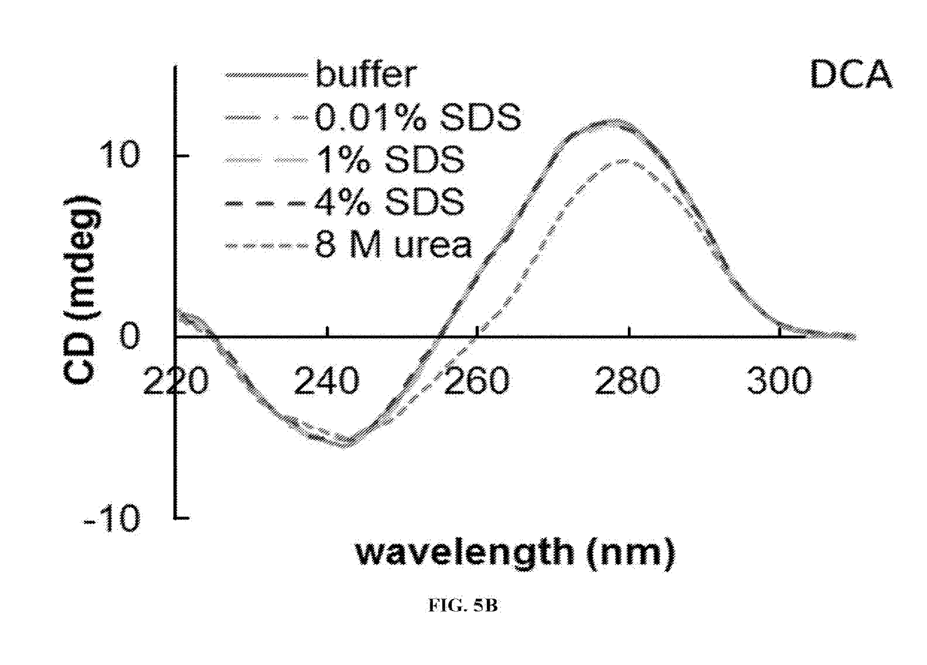

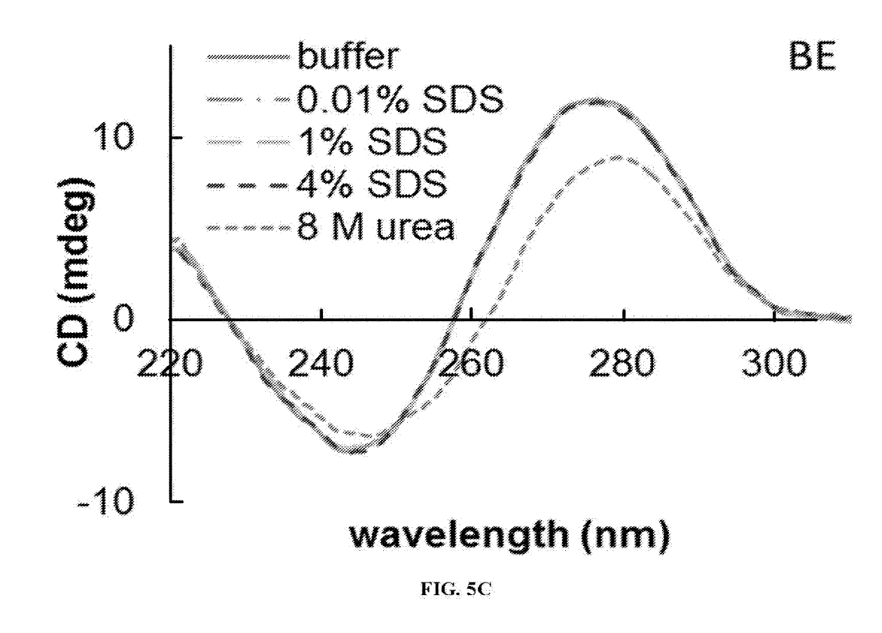

FIGS. 5A, 5B and 5C shows the CD spectra for the DIS (FIG. 5A), DCA (FIG. 5B) and BE (FIG. 5C) aptamers in the presence of 0, 0.01, 1, and 4% SDS, and 8 M urea.

As shown in FIGS. 5A, 5B and 5C, the CD spectra for each aptamer remain constant as the SDS concentration is increased from 0 to 4%. However, in the presence of 8 M urea, the CD signal undergoes a noticeable bathochromic shift and a slight decrease in intensity. Together, these data suggest that the aptamers are able to maintain their secondary structure in the presence of up to 4% SDS, which was not expected given that this concentration of SDS leads to denaturation of most proteins. Additionally, these results indicate that all three aptamers were likely to maintain their target-binding ability in the presence of up to 4% SDS.

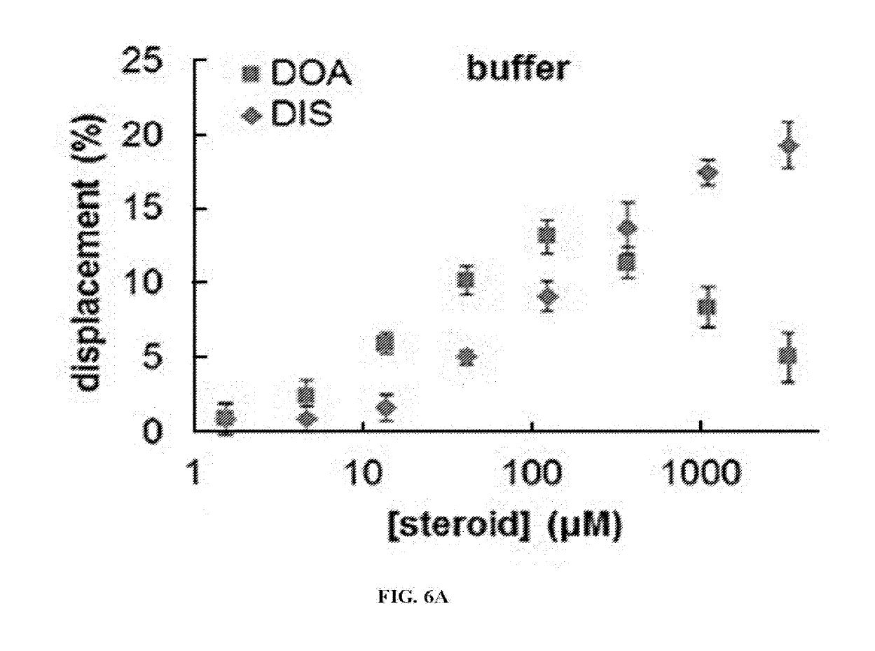

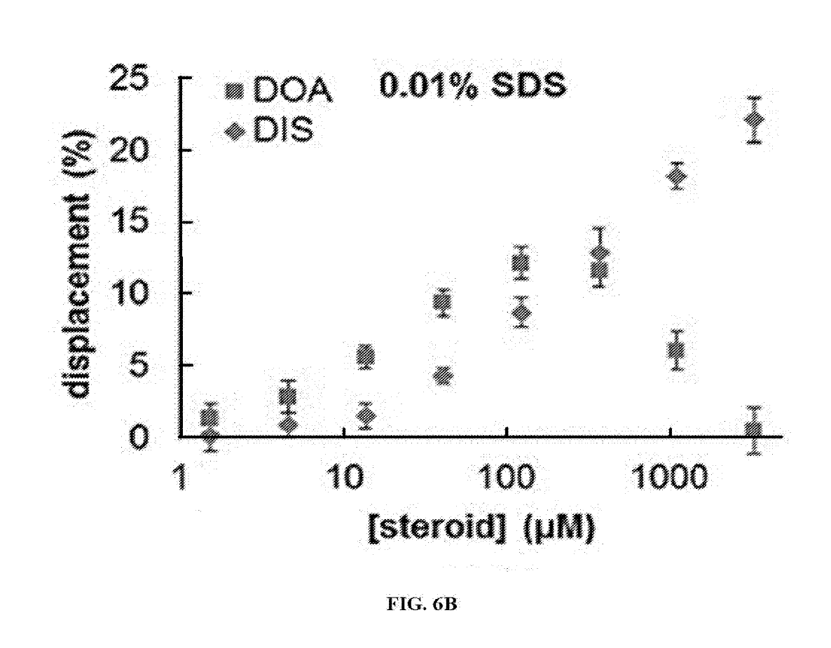

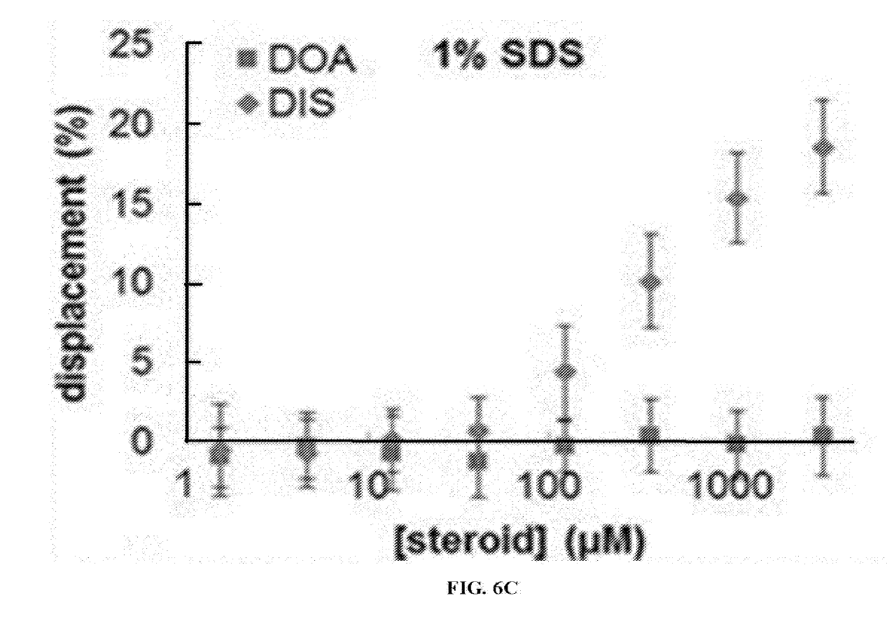

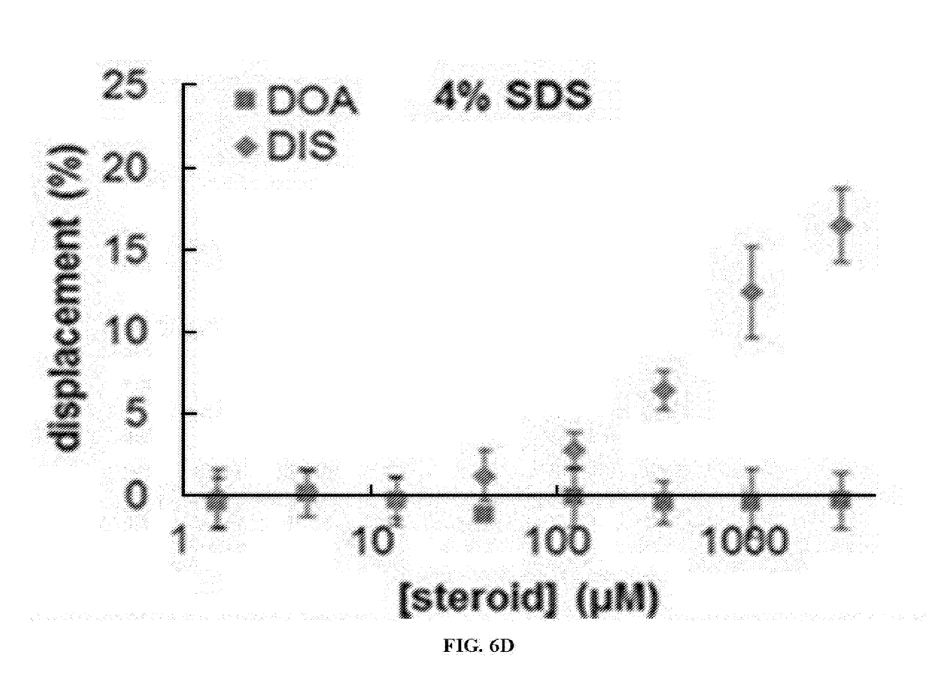

Modulating Target Selectivity. To determine whether a surfactant could be used to increase selectivity for hydrophilic ligands, the response of the DIS aptamer to both DIS and DOA in the presence of increasing concentrations of SDS was investigated. The data is shown in FIGS. 6A, 6B, 6C, and 6D, which are graphs of the percent displacement of the aptamer in the presence of one of the aptamer targets (DOA in squares, or DIS in diamonds), with no surfactant present (FIG. 6A), or with SDS present in a concentration of 0.01% (FIG. 6B), 1% (FIG. 6C), or 4% (FIG. 6D). The error bars represent the standard deviation of three independent trials.

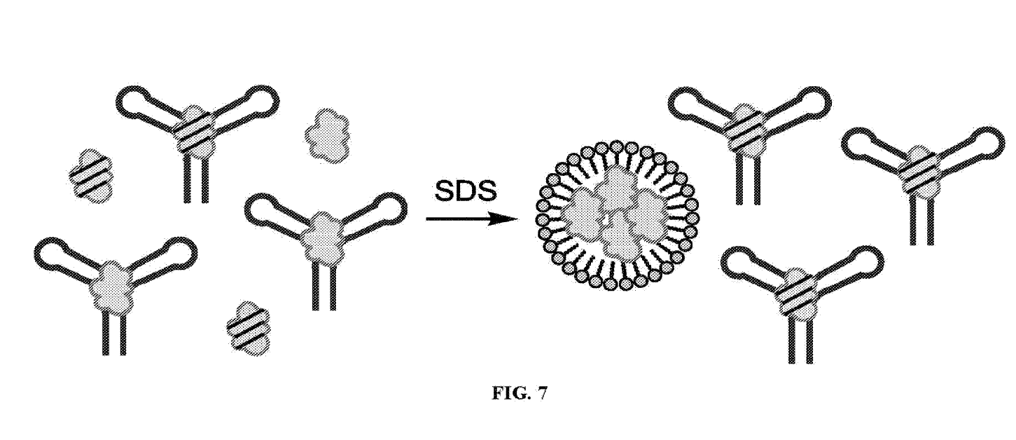

In buffer and 0.01% SDS, DOA was observed to bind to the DIS biosensor with slightly higher affinity than the DIS ligand. In both of these solutions, the fluorescence signal from DOA unexpectedly decreased at high ligand concentrations, possibly due to aggregation of the hydrophobic steroid. Upon increasing the SDS concentration to 1 or 4%, the biosensor shows no response to even mM concentrations of DOA, but shows only slightly attenuated binding to DIS. This switch in substrate binding preference presumably results from sequestration of the hydrophobic DOA in the micelles, whereas the hydrophilic DIS remains solvated by the aqueous phase. This is shown schematically in FIG. 7.

The data for FIGS. 6A-6D in tabular form for the average percent displacement (% dis.) and standard deviation (s.d.) for three trials using the DIS aptamer at various SDS concentrations with DOA or DIS as the ligand is shown below in Table 3:

TABLE 3
Displacement data for DOA and DIS at various concentrations of SDS.

| | | | | | | | 4% SDS | |
|---|---|---|---|---|---|---|---|---|
| | 0% SDS | | 0.01% SDS | | 1% SDS | | % | |
| | % dis. | s.d. | % dis. | s.d. | % dis. | s.d. | dis. | s.d. |
| [DOA] | | | | | | | | |
| (μM) | | | | | | | | |
| 3333 | 4.98 | 1.64 | 0.43 | 1.44 | 0.35 | 2.48 | −0.24 | 1.72 |
| 1111 | 8.34 | 1.33 | 6.01 | 1.40 | −0.20 | 2.12 | −0.43 | 2.07 |
| 370 | 11.38 | 1.04 | 11.52 | 1.34 | 0.40 | 2.28 | −0.39 | 1.33 |
| 123 | 13.11 | 1.10 | 12.12 | 1.60 | −0.29 | 1.62 | −0.09 | 1.77 |
| 41.2 | 10.15 | 0.92 | 9.38 | 0.13 | −1.27 | 2.23 | −1.09 | 0.46 |
| 13.7 | 5.93 | 0.74 | 5.57 | 0.77 | −0.73 | 2.31 | −0.29 | 1.48 |
| 4.57 | 2.32 | 1.10 | 2.80 | 0.84 | −0.26 | 2.09 | 0.17 | 1.40 |
| 1.52 | 0.83 | 1.06 | 1.34 | 1.15 | −0.97 | 1.87 | −0.40 | 1.54 |
| [DIS] | | | | | | | | |
| (μM) | | | | | | | | |
| 3333 | 19.27 | 1.55 | 22.07 | 1.46 | 18.54 | 2.94 | 16.49 | 2.23 |
| 1111 | 17.43 | 0.82 | 18.14 | 1.32 | 15.34 | 2.86 | 12.44 | 2.77 |
| 370 | 13.67 | 1.68 | 12.87 | 1.55 | 10.09 | 2.95 | 6.46 | 1.20 |
| 123 | 9.11 | 1.04 | 8.69 | 1.07 | 4.37 | 2.98 | 2.78 | 1.08 |
| 41.2 | 5.00 | 0.49 | 4.29 | 0.96 | 0.71 | 2.06 | 1.26 | 1.54 |
| 13.7 | 1.63 | 0.87 | 1.51 | 0.80 | 0.10 | 2.01 | −0.16 | 1.22 |
| 4.57 | 0.82 | 0.85 | 0.82 | 0.79 | −0.70 | 2.10 | 0.21 | 1.43 |
| 1.52 | 0.81 | 1.06 | 0.11 | 0.86 | −0.48 | 2.87 | −0.09 | 1.75 |

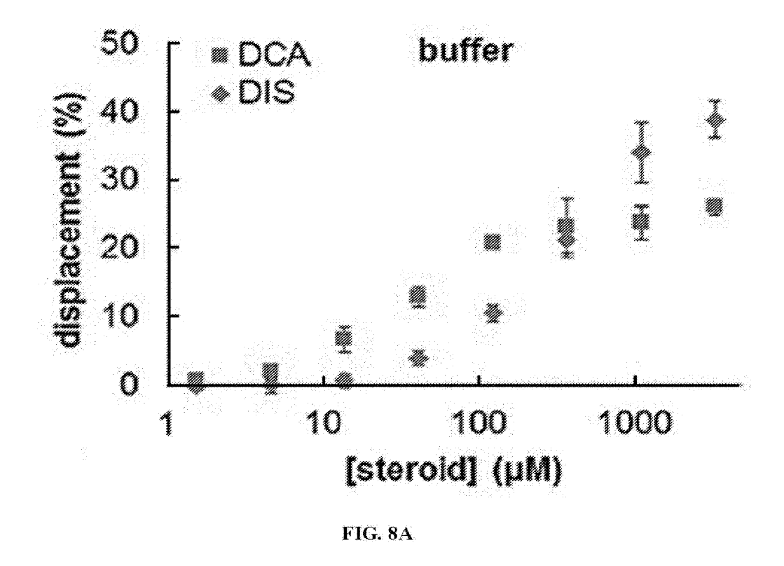

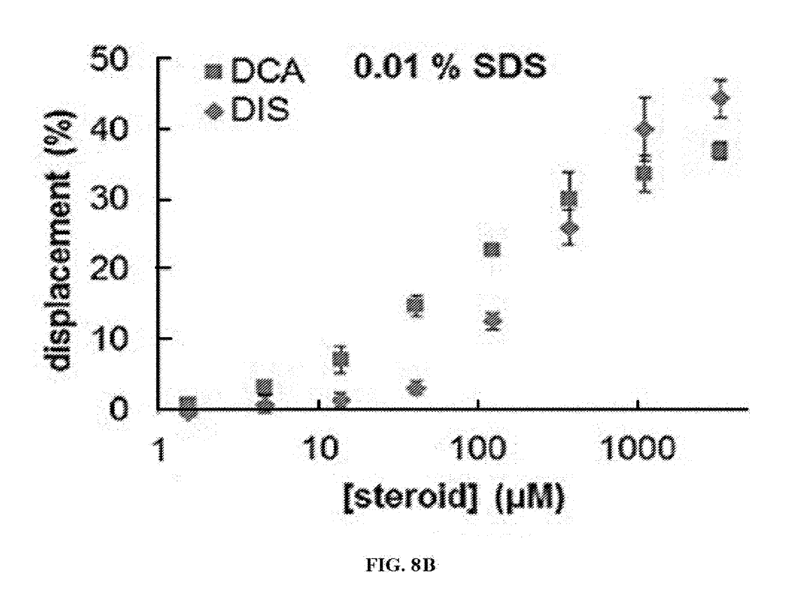

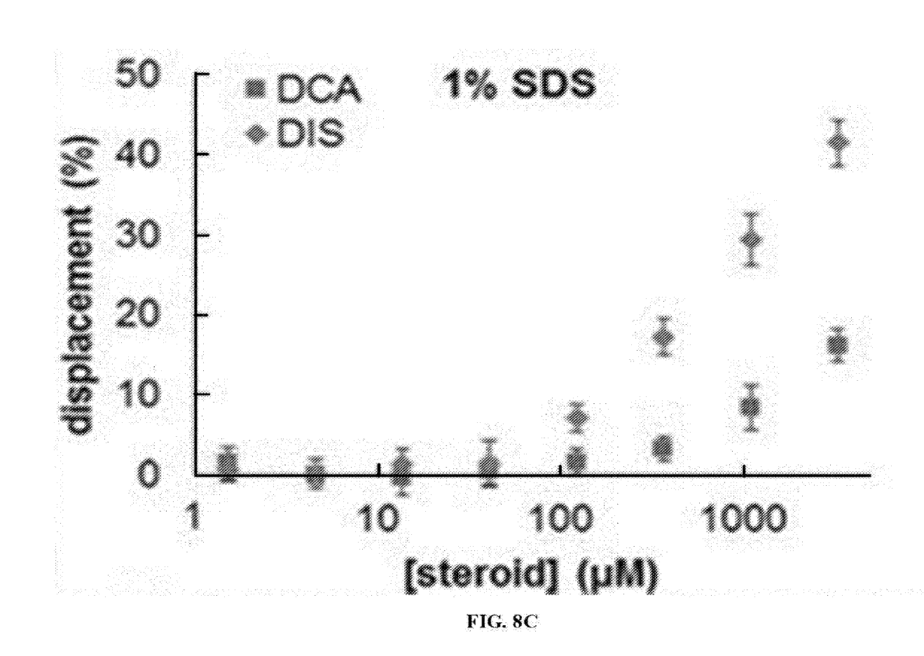

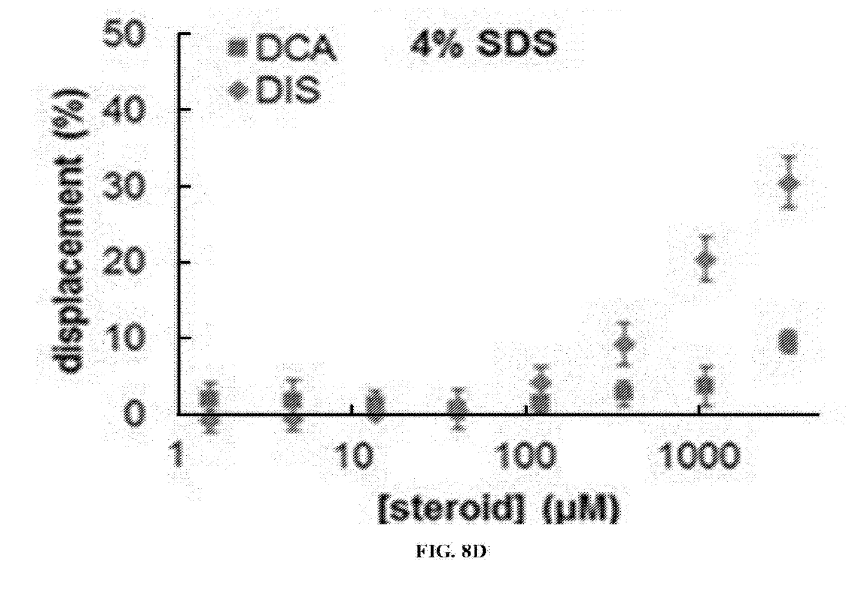

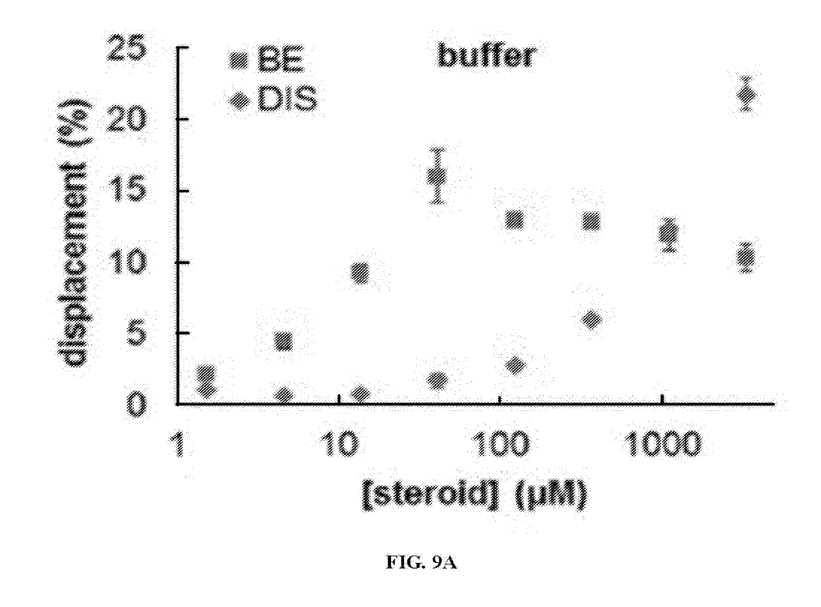

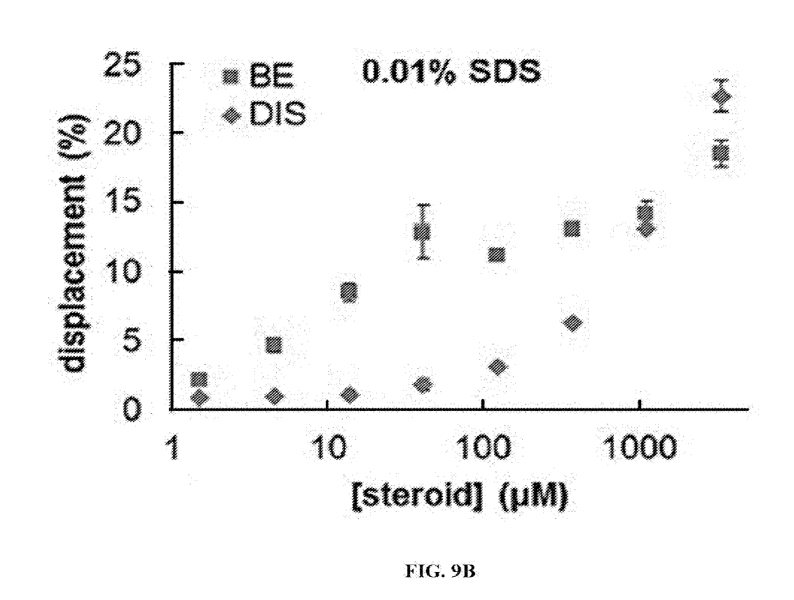

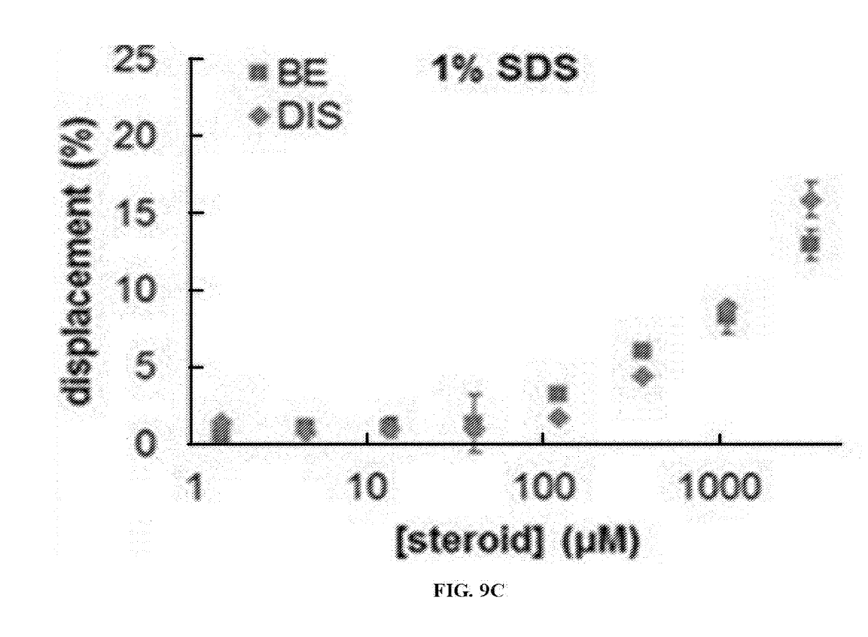

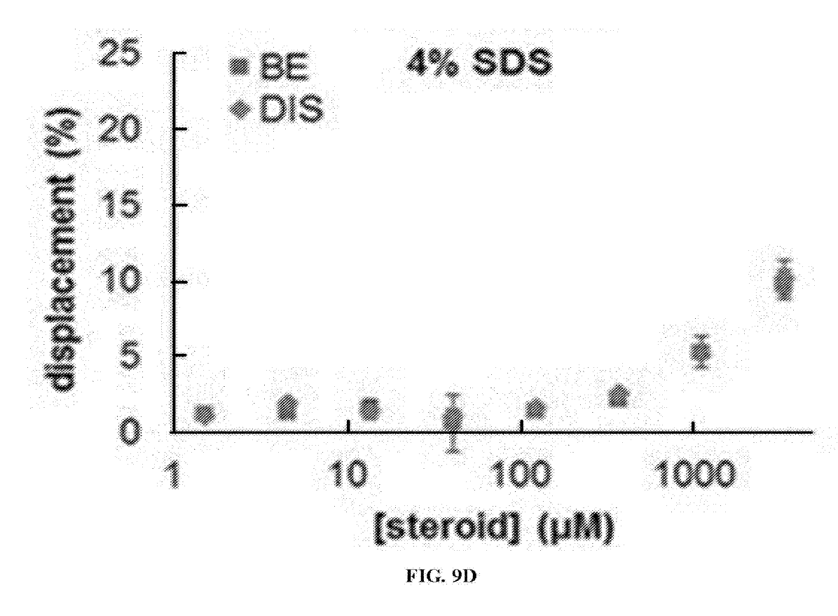

The impact of increasing SDS concentration on the substrate specificity of the DCA and BE biosensors was also investigated, and shown in FIGS. 8A-8D (DCA) and FIGS. 9A-9D (BE). FIG. 8A-8D show the fluorescence response of the DCA biosensor to DCA (squares) or DIS (diamonds) in (FIG. 8A) buffer, (FIG. 8B) 0.01% SDS, (FIG. 8C) 1% SDS, and (FIG. 8D) 4% SDS. Similarly, FIG. 9A-9D show the fluorescence response of the BE biosensor to BE (squares) or DIS (diamonds) in (FIG. 9A) buffer, (FIG. 9B) 0.01% SDS, (FIG. 9C) 1% SDS, and (FIG. 9D) 4% SDS. The error bars represent the standard deviation of three independent trials.

The data for FIGS. 8A-8D in tabular form for the average percent displacement (% dis.) and standard deviation (s.d.) for three trials using the DCA aptamer at various SDS concentrations with DCA or DIS as the ligand is shown below in Table 4:

TABLE 4
Displacement data for DCA and DIS at various concentrations of SDS.

| | | | | | | | 4% SDS | |
|---|---|---|---|---|---|---|---|---|
| | 0% SDS | | 0.01% SDS | | 1% SDS | | % | |
| | % dis. | s.d. | % dis. | s.d. | % dis. | s.d. | dis. | s.d. |
| [DCA] | | | | | | | | |
| (μM) | | | | | | | | |
| 3333 | 26.01 | 1.16 | 36.78 | 2.05 | 16.10 | 2.03 | 9.57 | 1.45 |
| 1111 | 23.66 | 2.50 | 33.52 | 3.15 | 8.37 | 2.74 | 3.68 | 2.64 |
| 370 | 23.18 | 3.93 | 29.87 | 4.37 | 3.22 | 1.47 | 2.78 | 1.64 |
| 123 | 20.66 | 0.81 | 22.72 | 6.70 | 1.59 | 1.62 | 1.47 | 1.13 |
| 41.2 | 12.89 | 1.51 | 14.77 | 2.40 | 0.40 | 1.69 | 0.77 | 2.58 |
| 13.7 | 6.54 | 1.87 | 7.10 | 2.91 | −0.36 | 2.15 | 1.37 | 1.78 |
| 4.57 | 1.99 | 1.11 | 3.19 | 1.30 | 0.17 | 1.79 | 1.83 | 2.62 |
| 1.52 | 0.59 | 1.04 | 0.70 | 1.99 | 1.52 | 2.00 | 1.93 | 2.11 |
| [DIS] | | | | | | | | |
| (μM) | | | | | | | | |
| 3333 | 38.73 | 2.63 | 44.36 | 4.34 | 41.45 | 2.84 | 30.43 | 3.29 |
| 1111 | 33.87 | 4.41 | 39.97 | 3.50 | 29.24 | 3.19 | 20.45 | 2.96 |
| 370 | 21.15 | 2.51 | 25.96 | 2.70 | 17.22 | 2.25 | 9.18 | 2.74 |
| 123 | 10.40 | 1.19 | 12.55 | 1.14 | 7.16 | 1.71 | 4.03 | 2.31 |
| 41.2 | 3.90 | 1.03 | 3.10 | 4.19 | 1.43 | 2.86 | 0.49 | 1.00 |
| 13.7 | 0.58 | 1.00 | 1.27 | 1.29 | 1.33 | 1.85 | 0.14 | 0.64 |
| 4.57 | −0.08 | 1.08 | 0.75 | 0.73 | 0.04 | 1.12 | −0.50 | 1.52 |
| 1.52 | −0.16 | 0.65 | −0.45 | 1.60 | 0.60 | 1.42 | −0.89 | 1.46 |

The data for FIGS. 9A-9D in tabular form for the average percent displacement (% dis.) and standard deviation (s.d.) for three trials using the BE aptamer at various SDS concentrations with BE or DIS as the ligand is shown below in Table 5:

TABLE 5
Displacement data for BE and DIS at various concentrations of SDS.

| | | | | | | | 4% SDS | |
|---|---|---|---|---|---|---|---|---|
| | 0% SDS | | 0.01% SDS | | 1% SDS | | % | |
| | % dis. | s.d. | % dis. | s.d. | % dis. | s.d. | dis. | s.d. |
| [BE] | | | | | | | | |
| (μM) | | | | | | | | |
| 3333 | 10.41 | 0.96 | 18.52 | 1.70 | 13.00 | 2.00 | 9.73 | 0.32 |
| 1111 | 11.97 | 1.03 | 14.05 | 0.24 | 8.24 | 0.30 | 5.31 | 0.27 |
| 370 | 12.84 | 0.23 | 13.01 | 0.33 | 6.04 | 0.64 | 2.16 | 0.28 |
| 123 | 12.89 | 0.36 | 11.25 | 0.29 | 3.25 | 0.05 | 1.46 | 0.10 |
| 41.2 | 15.93 | 1.87 | 12.83 | 0.51 | 1.34 | 0.32 | 0.65 | 0.15 |
| 13.7 | 9.27 | 0.63 | 8.59 | 0.04 | 1.08 | 0.02 | 1.54 | 0.47 |
| 4.57 | 4.50 | 0.51 | 4.72 | 0.12 | 1.06 | 0.11 | 1.35 | 0.12 |
| 1.52 | 2.11 | 0.20 | 2.15 | 0.06 | 0.60 | 0.10 | 1.18 | 0.07 |
| [DIS] | | | | | | | | |
| (μM) | | | | | | | | |
| 3333 | 21.70 | 1.11 | 22.68 | 0.56 | 15.86 | 0.91 | 10.21 | 0.67 |
| 1111 | 12.42 | 0.22 | 13.16 | 0.24 | 8.86 | 0.12 | 5.15 | 0.57 |
| 370 | 6.03 | 0.14 | 6.36 | 0.16 | 4.40 | 0.37 | 2.63 | 0.08 |
| 123 | 2.77 | 0.08 | 3.14 | 0.13 | 1.76 | 0.10 | 1.59 | 0.25 |
| 41.2 | 1.67 | 0.44 | 1.83 | 0.15 | 1.04 | 0.23 | 1.09 | 0.17 |
| 13.7 | 0.74 | 0.19 | 1.11 | 0.07 | 1.04 | 0.03 | 1.45 | 0.22 |
| 4.57 | 0.62 | 0.11 | 0.93 | 0.14 | 0.82 | 0.09 | 1.98 | 0.21 |
| 1.52 | 1.03 | 0.10 | 0.85 | 0.08 | 1.43 | 0.09 | 1.14 | 0.22 |

As can be seen in FIGS. 8A-8D, the DCA biosensor binds DCA with slightly higher affinity than DIS in buffer or 0.01% SDS. However, in the presence of micelles at 1 or 4% SDS, binding to DCA is attenuated, switching the preferred ligand to DIS. Such a dramatic reduction in DCA binding in the presence of micelles was initially surprising, as DCA has a charged carboxylate functional group, and thus would be expected to have some ability to remain solvated by the aqueous phase. However, DCA possesses an additional aliphatic chain relative to DIS, and the sulfate group of DIS contains a greater number of polar heteroatoms than the carboxylate of DCA. Thus, it is reasonable that the micelles sequester DCA, while leaving DIS free in solution.

In the case of the BE biosensor, the effect of SDS on substrate selectivity proved to be slightly more complex, as seen in FIGS. 9A-9D. At SDS concentrations below the CMC, the biosensor strongly favors BE, showing the highest affinity binding of all of the aptamer-ligand pairs. Above the CMC, the biosensor shows nearly equal binding to both DIS and BE. Increasing the concentration of SDS from 1 to 4% shows no appreciable effect on binding, which was surprising, as it was expected that BE would be strongly sequestered within the micelles due to its hydrophobicity. This anomalous observation may be attributed to the dynamic nature of ligand exchange between micelles, coupled with the high affinity of the aptamer for BE. However, despite some unexpected results, it was found that for each of the three biosensors, surfactant can be used to increase selectivity for hydrophilic over hydrophobic substrates.

In summary, disclosed herein is evidence that nucleic acid aptamers can retain their secondary structure and substrate binding capability in the presence of up to 4% surfactant. Anionic and non-ionic surfactants are particularly well-tolerated, whereas cationic and zwitterionic surfactants can compromise substrate binding, likely because the positively charged functional groups on the surfactant interact with the negatively charged backbone of the DNA. However, SDS and Triton X-100 are among the most commonly used surfactants in biochemical applications, and SDS in particular is known to readily denature antibody reagents. Thus, the ability of aptamers to maintain their function in the presence of both of these surfactants provides an additional competitive advantage relative to antibodies, and will significantly increase the scope of analytical applications for which aptamers can be employed.

It was also found that surfactant micelles can be used to modulate the substrate binding preferences of aptamers by selectively encapsulating more hydrophobic ligands. For all three aptamers tested, it was observed that the presence of SDS at concentrations above the CMC greatly diminishes or completely eliminates biosensor response to the more hydrophobic substrate. However, biosensor response to the hydrophilic substrate is only slightly attenuated. Thus, these studies establish surfactant addition as an effective method for increasing the substrate selectivity of DNA aptamers, which will enable the use of aptamers having non-ideal substrate selectivity for analytical applications where minimizing cross-reactivity is of critical importance.

### Example 2

SELEX Procedure. Amine-functionalized M-270 Dynabeads were purchased from Thermo Scientific. Sulfo-N-succinimidyl 4-(maleimidomethyl)cyclohexane-1-carboxylate sodium (SMCC crosslinker) was purchased from Chem Impex (cat no. 23033). PCR components were purchased from New England Biolabs. All DNA was purchased from the University of Utah DNA/Peptide Synthesis Core Facility, where it was synthesized using phosphoramidites and CPG cartridges from Glen Research. Fluorescence measurements were acquired using a Biotek Synergy Mx microplate reader with 2\,ex=495 nm and 2\,em=525 nm. All steps were performed at room temperature.

Bead Functionalization Protocol 1. Aliquots containing 1 mL of amine-functionalized Dynabeads (˜2×10⁹ beads/mL) were placed in a nonstick tube. The beads were washed twice with 1 mL PBS (pH=7.4). After each wash, the supernatant was removed using a magnetic separation stand (Dynal, Life Technologies). The beads were resuspended in PBS containing ˜1 mg/mL of the SMCC crosslinker and then incubated on a nutating mixer for 4 hours. The sample was placed on the magnetic separation stand for 1 min to remove the supernatant, followed by 3 washes with 1 mL PBS. After washing the beads 3 times, the beads were resuspended in 1 mL of PBS.

Denatured HSA (2 mg/mL) was prepared by dissolving HSA in PBS containing 1% SDS and 25 mM freshly prepared TCEP. The protein solution was heated to 95° C. for 10 min and cooled before addition to the SMCC-capped beads. The beads were again placed on the nutating mixer for 4 hours, followed by two washes with 1 mL PBS. A solution containing 1 M 2-mercaptohexanol in 1 mL of PBS was added to the beads to cap any remaining maleimide functional groups.

A sample of “blank” beads was also prepared by coupling the SMCC linker to the beads and capping with 2-mercaptohexanol as described above. In order to verify that HSA was attached to the beads, a solution containing an NHS-ester Cy3 dye was added to the HSA beads and the blank beads and their fluorescence was measured.

Bead Functionalization Protocol 2. Aliquots containing 1 mL of amine-functionalized Dynabeads (˜2×10⁹ beads/mL) were placed in a nonstick tube. The beads were washed twice with 1 mL PBS (pH=7.4). After each wash, the supernatant was removed using a magnetic separation stand (Dynal, Life Technologies). The beads were resuspended in PBS containing ˜1 mg/mL of the SMCC crosslinker and then incubated on a nutating mixer for 4 hours. The sample was placed on the magnetic separation stand for 1 min to remove the supernatant, followed by 3 washes with 1 mL PBS. After washing the beads 3 times, the beads were resuspended in 1 mL of PBS.

Denatured HSA (2 mg/mL) was prepared by dissolving HSA in PBS containing 1% SDS and 25 mM freshly prepared TCEP and added to the SMCC coated beads. The beads were again placed on the nutating mixer for 4 hours, followed by two washes with 1 mL PBS. A solution containing 1 M 2-mercaptohexanol in 1 mL of PBS was added to the beads to cap any remaining maleimide functional groups.

A sample of “blank” beads was also prepared by coupling the SMCC linker to the beads and capping with 2-mercaptohexanol as described above. In order to verify that HSA was attached to the beads, a solution containing an NHS-ester Cy3 dye was added to the HSA beads and the blank beads and their fluorescence was measured.

PCR amplification of initial ssDNA library. PCR was performed using a FAM-labeled forward primer (5′-/FAM/GCGCATACCAGCTTATTCAATT-3′) and a PEG-P20 reverse primer (5′-TTTTTTTTTTTTTTTTTTTT/Sp9/GCCGAGATTGCACTTACTATCT-3′) in order to facilitate strand separation. Taq polymerase, buffer, and dNTPs were purchased from New England Biolabs and reactant concentrations were done following their specifications. The PCR reaction was incubated at 95° C. for 3 min, and 17 cycles were performed heating the reaction to 95° C. for 30 sec, cooled to 51° C. for 30 sec, and incubated at 72° C. for 45 seconds. Following the final cycle, a final extension was performed by holding the reaction at 72° C. for 2 minutes. The double-stranded DNA product was purified using a PCR cleanup column (Qiagen). A gel extraction was then performed using an 8% denaturing PAGE gel (270 V, 35 min) to purify the desired single-stranded DNA fragment.

Selection. A single stranded DNA library having the sequence 5′-FAM-GCG-CAT-ACC-AGC-TTA-TTC-AAT-T-N₅₀-AGA-TAG-TAA-GTG-CAA-TCT-CGG-C-3′ was used to perform three separate and independent selections, where N₅₀ is a variable region comprising 50 nucleotides. Each selection utilized a method based on a previous SELEX protocol reported by Strehiltz and coworkers.

For all three selections, a negative selection was initially performed on the DNA library using the previously prepared blank beads to eliminate any sequences having affinity for the beads. 50 μL of the suspensions containing blank beads were placed in nonstick tubes, placed on a magnet for 4 min, and the supernatant was removed. A solution containing 200 pmol of the DNA library in 500 μL selection buffer was added to the beads, and they were incubated for 1 hour on a nutating mixer. The tubes again were placed on a magnet for 4 minutes, and the supernatant, which included DNA that was not bound to the blank beads, was then removed.

After obtaining the supernantant from the negative selection step, the three separate and independent selections were performed to identify DNA from the initial DNA library that binds to HSA-functionalized beads. For each of the three selections, 50 μL aliquots of the suspensions containing HSA-functionalized beads were placed in nonstick tubes, placed on a magnet for 4 min, and the supernatant was removed. The supernatants from the negative selection steps above were then added to the HSA-functionalized beads, and the samples were mixed on a nutating mixer for 1 hour. The supernatant was removed and the beads were washed five times with a selection buffer (PBS, pH 7.4, 1% SDS, 50 mM DTT), collecting all wash fractions. After washing the beads, remaining DNA was subsequently eluted from the HSA-functionalized beads using either a heat elution or a ligand elution, as disclosed in more detail below. The eluted DNA was then purified using Qiagen minelute columns, and any residual protein was digested using proteinase K at 37° C. for 1 hour followed by incubation at 95° C. for 20 min to denature the enzyme. The resulting library of nucleic acids that was eluted from the HSA-functionalized beads was subjected to 17 cycles of PCR followed by gel purification. The purified nucleic acids were then used for another round of SELEX, where the nucleic acids were again added to HSA-functionalized beads, the beads were washed to remove unbound DNA, the bound DNA was eluted, the eluted DNA was amplified with PCR, and the amplified DNA was purified. For each of the three selections, multiple rounds of SELEX were performed.

For the first selection, a heat elution process was utilized to elute DNA that was bound to the HSA-functionalized beads for each round of SELEX that was performed. Specifically, after adding the DNA to the HSA-functionalized beads and then washing the beads, 500 μL of elution buffer (PBS, pH 7.4, 1% SDS, 10 mM EDTA, 3.5 M urea) was added to the sample followed by incubation at 95° C. for 8 min. The supernatant was removed, and the elution was repeated. The eluent from the two elution steps was combined.

For the second and third selections, ligand elution processes were utilized to elute DNA from the HSA-functionalized beads for each round of SELEX that was performed. Specifically, after adding the DNA to the HSA-functionalized beads and washing the beads, 500 μL of a solution of denatured HSA (2 mg/mL) in the selection buffer was added to the beads. The SDS concentration was maintained at 1% by adding 14 μL 20% SDS to 1 mL denatured protein. The samples were then incubated on a nutating mixer for 1 hour, and the supernatant collected. The elution was then repeated, and the eluents were combined.

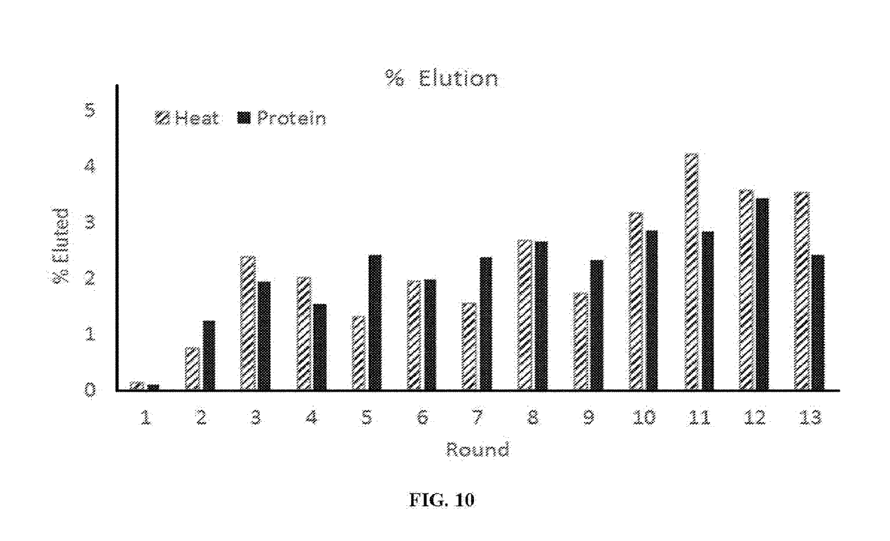

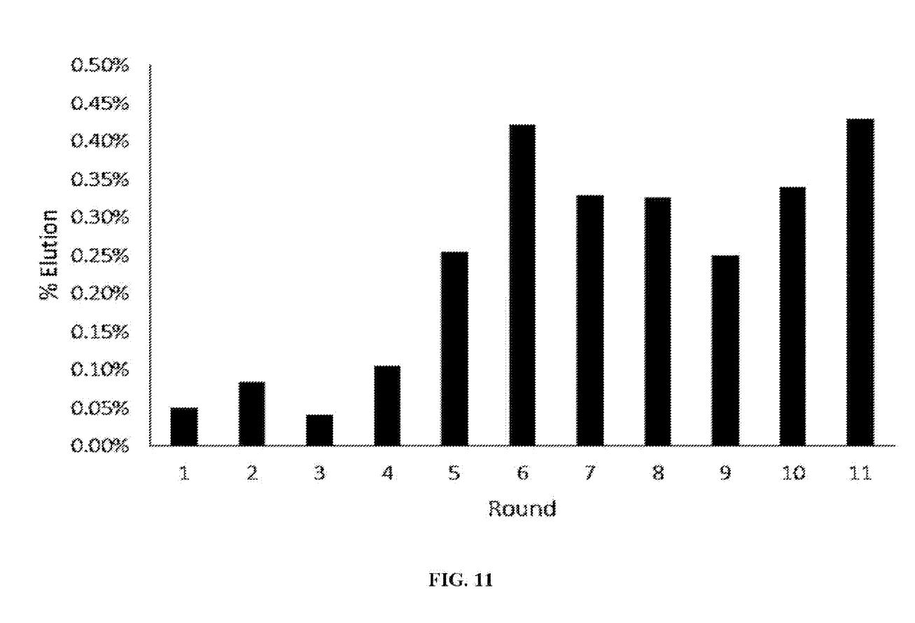

The total fluorescence of the eluent from each round of SELEX was measured and compared to the total amount of fluorescence added to the HSA-functionalized beads for that round of SELEX so as to assess the approximate percentage of DNA eluted in that round of SELEX. The results from the first and second selections are shown in FIG. 10, which shows the percentage of DNA eluted in each round of SELEX using the heat elution (cross-hatched bars) and ligand elution (black bars) protocols. The results of the third selection are shown in FIG. 11, which shows the percentage of DNA eluted in each round SELEX using the ligand elution protocol.

The DNA obtained from the final round of SELEX for each of the three selections was then amplified with PCR, and was cloned into a DNA plasmid. Top10 E. coli were transformed through the uptake of this vector, and colonies were grown. We arbitrarily selected 20 E. coli colonies transformed with DNA recovered from the first selection, 20 E. coli colonies transformed with DNA recovered from the second selection and 40 E. coli colonies transformed with DNA recovered from the third selection. We isolated the DNA from these colonies for Sanger sequencing. Following sequencing, the 20 DNA sequences from the first selection were labeled H1 through H20, the 20 DNA sequences from the second selection were labeled P1 through P20, and the 40 DNA sequences from the third selection were labeled 1-40.

Using Multalin software, we compared all 80 sequences from the three selections, grouped them based on homology, and sorted the sequences into multiple groups. Representative sequences were arbitrarily selected from each of these groups to test to determine whether they would bind HSA under denaturing and optionally reducing conditions using gel-shift and pull-down assays, as described below. These representative sequences included the sequences shown in Table 6:

TABLE 6
Sequences of various nucleic
acids obtained from selections 1-3

| SEQ | | |
|---|---|---|
| ID NO | Name | Sequence (5′→3′) |
| 7 | H1 | GCG CAT ACC AGC TTA TTC AAT TAC |
| | | GCT CAG GAC TAA AAA TGG GCG TGA |
| | | CCA TGA GCT CAG TAT GGG CGT TAC |
| | | AGA TAG TAA GTG CAA TCT CGG C |
| 8 | H2 | GCG CAT ACC AGC TTA TTC AAT TTC |
| | | AAA GGC TGA CCG GAT GGG CTT CGT |
| | | GAC CGG GTA CGC CAC TGC CTC GTG |
| | | AGA TAG TAA GTG CAA TCT CGG C |
| 9 | H3 | GCG CAT ACC AGC TTA TTC AAT TGA |
| | | TCC ATA CAA GGG TAG GTT GAC TTC |
| | | CGC CCA AGG GGG CGA GGC CGT GAG |
| | | ATA GTA AGT GCA ATC TCG GC |
| 10 | H5 | GCG CAT ACC AGC TTA TTC AAT TAG |
| | | GAA CGC ACC CGA GTG TTG CGC GAG |
| | | CCA GTG GCT GGT TGG CCC GTA TAA |
| | | GAT AGT AAG TGC AAT CTC GGC |
| 11 | H6 | GCG CAT ACC AGC TTA TTC AAT TGC |
| | | GTA CCT AGG ATT TAC TTT CTC AAG |
| | | ATA TTT GGA GTG ACA AGT GGC ATA |
| | | AGA TAG TAA GTG CAA TCT CGG C |
| 12 | P1 | GCG CAT ACC AGC TTA TTC AAT TGC |
| | | ATA GTC ATG CGG GAA TAG TTT TCG |
| | | ATG AGG GCT ATT GGA GGC CCC AGC |
| | | AGA TAG TAA GTG CAA TCT CGG C |
| 13 | 19 | GCG CAT ACC AGC TTA TTC AAT TAA |
| | | CGT GTA GAG GTG TTC ACG CAC CAC |
| | | GCC GAC TGT GGT TGA CCC ACA GTT |
| | | AGA TAG TAA GTG CAA TCT CGG C |
| 14 | 35 | GCG CAT ACC AGC TTA TTC AAT TCG |
| | | GTA CGT GAC GCA AAA AAG GTC GGG |
| | | CGG CTG ACA TGG CAC CTC GTC TAA |
| | | AGA TAG TAA GTG CAA TCT CGG C |
| 15 | 36 | GCG CAT ACC AGC TTA TTC AAT TCG |
| | | GAT AGA CAA CAT GGG GAA GAA CAG |
| | | TGT CTA GTC GTG GGT TGC TCT GTA |
| | | AGA TAG TAA GTG CAA TCT CGG C |
| 16 | 40 | GCG CAT ACC AGC TTA TTC AAT TCG |
| | | GTA GTG CAG CGG CTT GAA ATC AGC |
| | | CGT GCT TTC GGG ACG GAA CTA CTC |
| | | AGA TAG TAA GTG CAA TCT CGG C |

Gel Shift Assays. 28 different nucleic acids were produced having 5′ fluorescein labels according to known methods. Specifically, nucleic acids were synthesized using solid state synthesis where a fluorescein phosphoramidite (Glen Research, part number 10-5901) was used to attach a fluorescein label to the 5′ end of the nucleic acid. The 28 nucleic acids included all of the nucleic acids listed in Table 6 above.

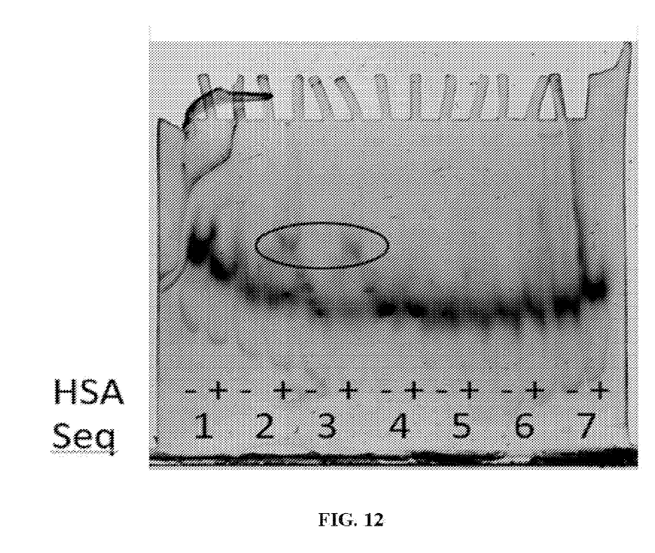

Gel shift assays were then performed to determine which of these nucleic acids would bind to HSA under denaturing and reducing conditions. Specifically, 40 nmol of the fluorescein labeled DNA was incubated with 3 μM denatured HSA in PBS (pH=6) containing 50 mM fresh DTT and 1% SDS at room temperature for 1 hr. The samples were then run on an 8% nondenaturing gel (PBS pH=6, 0.1% SDS) at 100 V for 1 hour at 4° C. The gels were imaged using a Typhoon FLA 9500 gel imager using a 473 nm laser and an LPB filter. FIG. 12 is a representative gel showing the results of the gel shaft assays for the fluorescein labeled forms of the following nucleic acids: 35 (lane 1), 36 (lane 2), 40 (lane 3), H1 (lane 4), H2 (lane 5), H3 (lane 6) and H5 (lane 7). As can be seen in FIG. 12, aptamers 36 and 40 (SEQ ID NOS 15 and 16) bound to the DNA even under denaturing and reducing conditions. Although not shown in FIG. 12, the gel shift assays also revealed that three other sequences bind to HSA under denaturing and reducing conditions, including aptamers 19, P1 and H6.

Pull-Down Assays. After performing the gel shift assays, the sequences that were identified in that assay as being capable of binding to HSA under denaturing and reducing conditions were coupled to agarose beads, and the modified beads were tested to determine whether they could be used to pull down HSA from samples under denaturing and reducing conditions. Specifically, aptamers 19, 36, 40, P1 and H6 were selected for testing.

To generate the modified agarose beads, aptamers 19, 36, 40, P1 and H6, as well as a control DNA sequence having 20 Thymine residues (i.e., a T-20), were first modified to include a thiol group coupled to the 5′ end of the DNA with a 6-mer of polyethylene glycol as a linker between the nucleic acid and the thiol group. Specifically, the T-20 DNA (SEQ ID NO: 17) and nucleic acids 19, 36, 40, P1 and H6 (SEQ ID NOS: 13, 15, 16, 12 and 11) were synthesized using solid state synthesis, where at the 5′ end of the nucleic acid a Spacer Phosphoramidite 18 (Glen Research, part number 10-1918) was used to add the 6-mer polyethylene glycol linker, and then a Thiol-Modifier 6 S—S phosphoramidite (Glen Research, part number 10-1936) was used to attach a thiol modifier to the end of the polyethylene glycol linker. These modifications produced DNA having the following general formula: DMTO-(CH₂)₆—S—S—(CH₂)₆—PO₃—(OCH₂CH₂)₆—PO₃-DNA.

The modified nucleic acids were subsequently prepared for attachment to agarose beads by cleaving the dithiol in 0.18 M phosphate buffer (pH=8.0) and 100 mM DTT for 1 hour, so as to produce “cleaved” DNA having the following general formula: 5-(CH₂)₆—PO₃—(OCH₂CH₂)₆—PO₃-DNA. The modified DNA was then purified using an Amicon Ultra 10 kDa size exclusion filter.

Thermofischer Sulfolink agarose resin, which includes terminal iodoacetal groups, was warmed to room temperature and 800 μL were transferred to a centrifuge column to give a bed volume of 400 μL. The solvent was removed with brief centrifugation, and the resin was washed with four bed volumes of coupling buffer (1.6 mL). The agarose beads were then reacted with the thiolated nucleic acids to functionalize the beads. Specifically, 200 pmol of the “cleaved” DNA was diluted in coupling buffer (50 mM Tris (pH=8.5) containing 5 mM EDTA) and added to the resin at room temperature, whereupon the thiol groups of the thiolated nucleic acids reacted with the iodoacetal groups of the beads, thereby covalently coupling the nucleic acids to the resin.

The resin was placed on a nutating mixer for 15 min then stationary for 30 min, and the supernatant was removed. 700 μL of PBS (pH=7) containing 0.1% SDS was then added to the resin and the samples were mixed on a nutating mixer for 30 min followed by 15 minutes of stationary incubation. The resin was then washed 3 times with 700 μL 0.1% SDS in PBS then 2 times with 700 μL coupling buffer. The unreacted groups were capped by adding 2 μL 6-mercapto-1-hexanol in 400 μL coupling buffer. The resin was mixed on a nutating mixer for 15 min then stationary for 30 min. The resin was washed 3 times with 700 μL coupling buffer to remove residual MSH.

After preparing the modified agarose, HSA (2 mg/mL) was denatured in 1% surfactant and 50 mM DTT or 25 mM TCEP in PBS (pH=7.4) for 1 hour. The binding buffers consisted of 50 mM freshly prepared DTT or 25 mM TCEP, and 1% surfactant in 1×PBS buffer (pH=7.4).

The beads were equilibrated with 3 bed volumes of the binding buffer used during the pull-down prior to protein addition. Protein (40 μg/mL) was then added to the columns and placed on a nutating mixer for 45 min followed by 15 minutes of stationary incubation. The supernatant was then collected via centrifugation. The resin was then washed twice with 700 μL binding buffer, collecting the flow-through each time. Bound protein was then eluted using 300 μL 4 M urea with 0.1% surfactant and repeated to recover all bound protein. For each elution, the column was placed on a nutating mixer for 30 min. The A280 was then measured for each aliquot to determine the percent bound.

The columns were regenerated by rinsing with 6 bed volumes of 1 M NaCl followed by 2 bed volumes of PBS. They were then stored in PBS in the refrigerator.

The aliquots were concentrated using a 10 kDa Amicon spin filter. The samples were run on a NuPAGE™ Novex™ 4-12% Bis-Tris protein gel at 200 V for 40 min and stained with Spyro Ruby. The gels were visualized with a Typhoon FLA 9500 gel imager using a 473 nm laser and an LPG filter.

The results of the pull down assay are shown in Table 7, below (the % HSA bound by aptamer 36 is not shown because the tube with aptamer 36 was accidentally spilled. However, as discussed above, FIG. 12 shows that aptamer 36 binds HSA).

TABLE 7
The amount of HSA bound by each aptamer.

| Sequence | % HSA Bound |
|---|---|
| T-20 DNA | 0.63% |
| 19 | 10.31% |
| 40 | 10.28% |
| P1 | 12.70% |
| H6 | 10.04% |

This experiment demonstrates that the aptamer sequences generated via selection are capable of being immobilized on a bead and binding to HSA in solution under denaturing and reducing conditions. The lack of significant pull-down using a control T-20 sequence demonstrates that the binding of HSA is a specific property of the selected aptamers, and not a general property of DNA molecules.

### Example 3

RNA aptamers. The sulforhodamine B RNA aptamer developed by Tsien and coworkers (Babendure, J. R.; Adams, S. R.; Tsien, R. Y., Aptamers Switch on Fluorescence of Triphenylmethane Dyes, Journal of the American Chemical Society 2003, 125 (48), 14716-14717) was used to test RNA aptamer function in denaturing and/or reducing conditions. All buffers contained 100 mM KCl, 5 mM MgCl₂, and 10 mM HEPES (pH=7.4). For experiments testing function in the presence of Tween 80 and/or DTT, the solutions contained 1% Tween 80 and/or 50 mM DTT. A 95 μL solution was prepared containing 3.3 μM RNA in buffer. 5 μL of solution containing 200 μM Patent Blue V (PBV) was then added to the RNA solution (10 μM final ligand concentration). Blank solutions were prepared containing 10 μM PBV in each of the buffer conditions. The reactions were then incubated at 25° C. for 20 min and the fluorescence was recorded at 665 nm using λₑₓ=635 nm as measured using a Biotek Synergy MX plat reader. The fluorescence increase was calculated using the ratio of the solutions containing RNA to PBV free in solution. The data is shown in Table 8, below, where the fluorescence enhancement represents the increase in fluorescence of the PBV ligand in the presence of the RNA aptamer. This increase is indicative of binding of the ligand to the aptamer.

TABLE 8
The fluorescence enhancement observed in each solution after addition
of the aptamer.

| | Fluorescence |
|---|---|
| buffer | enhancement |
| HEPES Buffer | 13.7 ± 1.9 |
| 1% Tween 80 | 4.57 ± 0.21 |
| 50 mM DTT | 13.9 ± 1.6 |
| 1% Tween 80 & 50 mM DTT | 5.04 0.30 |

The target compound binds to PBV, which is relatively hydrophilic and doesn't bind in micelles. Tween 80 was used for these experiments, as the aptamer requires Mg²⁺, which precipitates in SDS. The values in the table show the fluorescence enhancement when RNA is added to the solution. The PBV dye is fluorogenic and known to increase fluorescence upon binding to the aptamer.

This data shows that an RNA aptamer can bind to its target compound in the presence of a surfactant, a reductant, and both a surfactant and a reductant.

### Example 4

HSA binding with DTT. Bead functionalization: Aliquots containing 1 mL of amine-functionalized Dynabeads (˜2×10⁹ beads/mL) were placed in a nonstick tube. The beads were washed twice with 1 mL PBS (pH=7.4). After each wash, the supernatant was removed using a magnetic separation stand (Dynal, Life Technologies). The beads were resuspended in PBS containing ˜1 mg/mL of the SMCC crosslinker and then incubated on a nutating mixer for 4 h. The sample was placed on the magnetic separation stand for 1 min to remove the supernatant, followed by 3 washes with 1 mL PBS.

Thiolated nucleic acid 36 (described above) was prepared for attachment to the beads by cleaving the dithiol in 0.18 M phosphate buffer (pH=8.0) and 100 mM DTT for 1 h. The DNA was then purified using a Amicon Ultra 10 kDa size exclusion filter. The cleaved DNA was resuspended in PBS and added to the SMCC-functionalized beads. The beads were again placed on the nutating mixer for 4 h, followed by two washes with 1 mL PBS. A solution containing 1M 2-mercaptohexanol in 1 mL of PBS was added to the beads to cap any remaining maleimide functional groups. A sample of “blank” beads were also prepared by coupling the SMCC linker to the beads and capping with 2-mercaptohexanol as previously described.

Pull-down. HSA (2 mg/mL from Gemini) was denatured in 1% surfactant (either Tween 80 or SDS) and 50 mM DTT in PBS (pH=7.4) for 1 hr. The binding buffers consisted of 50 mM freshly prepared DTT. The beads were equilibrated with 2 mL of the binding buffer used during the pull-down prior to protein addition. Protein (40 μg/mL) was then added to the columns and these were placed on a nutating mixer for 1 hr. The samples were then placed on the magnetic separation stand for 4 min, and the supernatant was removed. The beads were then washed twice with 1 mL binding buffer, collecting the supernatant each time. Bound protein was eluted using 300 μL of 4M urea with 0.1% surfactant and repeated to recover all bound protein. For each elution, the beads were incubated at 95° C. for 10 min. The beads were regenerated by rinsing with 1 mL of 1M NaCl followed by 2 mL of PBS. They were then stored in PBS in the refrigerator.

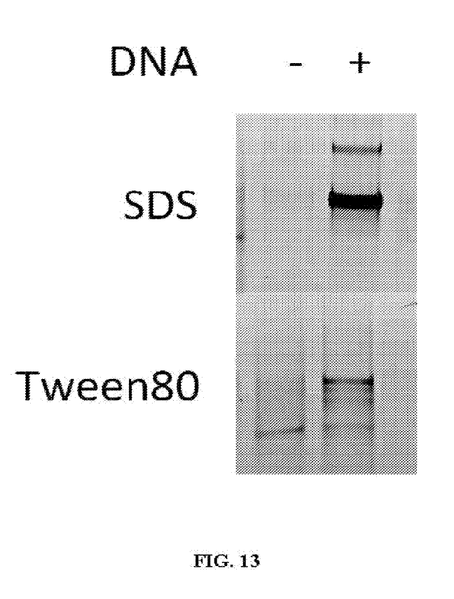

The aliquots were then concentrated using a 10 kDa Amicon spin filter. The samples were run on a NuPAGE™ Novex™ 4-12% Bis-Tris protein gel at 200 V for 40 min and stained with Spyro Ruby. The gels were visualized with a Typhoon FLA 9500 gel imager using a 473 nm laser and an LPG filter. FIG. 13 is a gel showing the results of the pull down (using nucleic acid 36) of HSA denatured with SDS (upper image) or Tween 80 (lower image) and reduced with DTT. The left lane of each gel image shows control “blank” beads with no DNA; the right lane of each gel image shows beads having the aptamer sequence attached.

As seen in FIG. 13, the DNA aptamer is able to bind to HSA in the presence of either SDS or Tween 80 and DTT. Notably, the aptamer was selected in the presence of SDS and is subsequently able to bind to its target compound in the presence of either Tween 80 or SDS as the surfactant and DTT as the reductant.

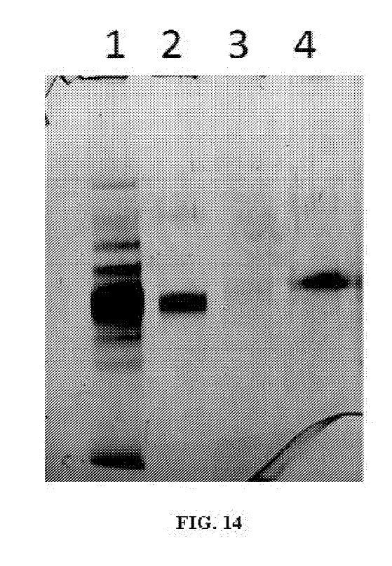

FIG. 14 is a gel showing the results of the selective pull down of HSA from a human plasma sample, which was denatured with Tween 80 and reduced with DTT. In FIG. 14, lane 1 is a control sample of human plasma that was denatured and reduced using Tween 80 and DTT; lane 2 is a control sample containing HSA which was denatured and reduced using Tween 80 and DTT; lane 3 is a sample arising from the eluent when human plasma is contacted with blank control beads (no aptamer) exposed to both surfactant and reductant; and lane 4 is a sample arising from the eluent when human plasma is contacted with aptamer 36-functionalized beads exposed to both surfactant and reductant. The ability of the aptamer 36-functionalized beads to purify HSA from a complex human plasma mixture demonstrates the selectivity of aptamer 36 in the intended application. As seen in FIG. 14, the DNA aptamer is able to bind to either purified HSA or to HSA found in human plasma (i.e. in a mixture of plasma proteins), in the presence of surfactant (Tween 80) and reductant (DTT).

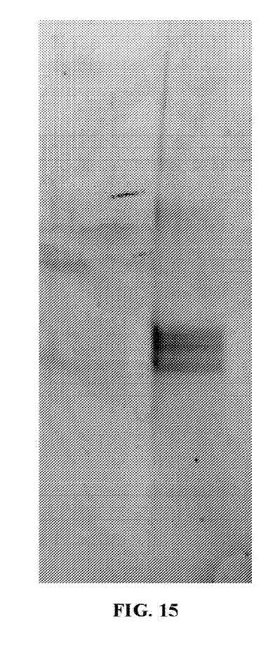

FIG. 15 is a gel showing the results of the selective pull down of HSA from a human plasma sample using aptamer 36-functionalized beads (as described above), which was denatured with Empigen BB and reduced with DTT. In FIG. 15, the left lane is a sample arising from the eluent when human plasma is contacted with blank control beads (no aptamer) exposed to both the surfactant and the reductant; the right lane is a sample arising from the eluent when human plasma is contacted with aptamer 36-functionalized beads exposed to both the surfactant and the reductant. The ability of the aptamer 36-functionalized beads to purify HSA from a complex human plasma mixture again demonstrates the selectivity of aptamer 36, here in the presence of a zwitterionic sufactant. As seen in FIG. 15, the aptamer 36 is able to bind to HSA found in human plasma (i.e. in a mixture of plasma proteins), in the presence of the surfactant (Empigen BB) and the reductant (DTT).

### Example 5

HSA binding with TCEP. HSA (2 mg/mL from Gemini) was denatured in 1% Tween 80 and 25 mM tris(2-carboxyethyl)phosphine (TCEP) in PBS (pH=7.4) for 1 hr. The binding buffers contained 1% Tween 80 and 25 mM TCEP in PBS (pH=7.4). Aptamer 36-functionalized prepared as described above were equilibrated with 2 mL of the binding buffer prior to protein addition. Protein (100 μg/mL) was then added to the tubes and placed on a nutating mixer for 1 hr. The samples were then placed on the magnet for 4 min, and the supernatant was removed. The beads were then washed twice with 1 mL binding buffer, collecting the supernatant each time. Bound protein was then eluted using 500 μL 4M urea with 0.1% Tween 80 and repeated to recover all bound protein. For each elution, the beads were incubated at 95° C. for 10 min. The beads were regenerated by rinsing with 1 mL of a solution containing 7M Urea, 2M thiourea, and 0.01% SDS followed by 2 mL of PBS. They were then stored in PBS in the refrigerator.

The aliquots were then concentrated using a 10 kDa Amicon spin filter. The samples were run on a NuPAGE™ Novex™ 4-12% Bis-Tris protein gel at 200 V for 40 min and stained with Spyro Ruby. The gels were visualized with a Typhoon FLA 9500 gel imager using a 473 nm laser and an LPG filter.

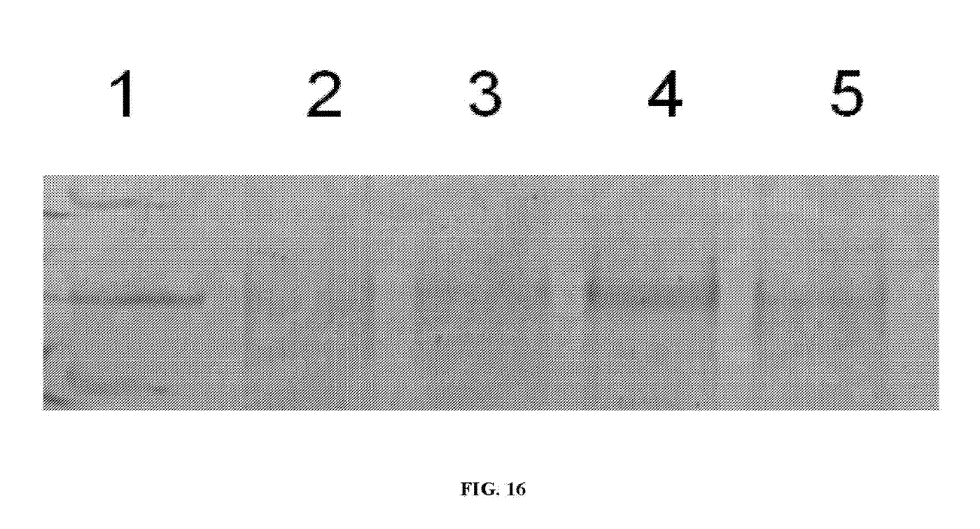

FIG. 16 is a gel showing the results of the pull down of HSA using aptamer 36-functionalized beads in the presence of Tween 80 and with TCEP as the reductant. In FIG. 16, lane 1 is a sample of eluent from the HSA control (no beads or surfactant or reductant present); lanes 2 and 3 are samples of eluent from HSA and surfactant and reductant but with no beads present; and lanes 4 and 5 are samples of eluent from HSA and aptamer 36-functionalized beads exposed to both surfactant and reductant.

As seen in FIG. 16, the aptamer 36-functionalized beads are able to bind to HSA in the presence of surfactant and TCEP as the reductant.

### REFERENCES

Each of the following citations is fully incorporated herein by reference in its entirety.
R. A. Weinberg, The Biology of Cancer, Garland Science, New York, N.Y. (2007).
T. R. Golub, D. K. Sloinim, P. Tamayo, C. Huard, M. Gaasenbeek, J. P. Mesirov, H. Coller, M. L. Loh, J. R. Downing, M. A. Caligiuri, C. D. Bloomfield, and E. S. Lander, “Molecular classification of cancer: Class discovery and class prediction by gene expression monitoring,” Science 286, pp. 531-537 (1999).
J. Lu, G. Getz, E. A. Miska, E. Alvarez-Saavedra, J. Lamb, D. Peck, A. Sweet-Cordero, B. L. Ebert, R. H. Mak, A. A. Ferrando, J. R. Downing, T. Jacks, H. R. Horvitz, and T. R. Golub, “MicroRNA expression profiles classify human cancers,” Nature 435, pp. 834-838 (2005).
Sung-Min Ahn and Richard J. Simpson, “Body fluid proteomics: prospects for biomarker discovery,” Proteomics Clin. Appli. 1, pp. 1004-1015 (23 Apr. 2007).
N. L. Anderson and N. G. Anderson, “The Human Plasma Proteome,” Mol. & Cell. Proteomics, 1(11), pp 845-867 (2002).
P. G. Righetti, A. Castagna, B. Herbert, and G. Candiano, “How to bring the “unseen” proteome to the limelight via electrophoretic pre-fractionation techniques,” Bioscience Reports 25, pp. 3-17 (2005).
G. L. Corthals, V. C. Wasinger, D. F. Hochstrasser, and J.-C. Sanchez, “The dynamic range of protein expression: A challenge for proteomic research,” Electrophor. 21, pp. 1104-1115 (2000).
G. L. Hortin and D. Sviridov, “The dynamic range problem in the analysis of the plasma proteome,” J. Proteomics 73, pp. 629-636 (2010).
N. L. Anderson, A. S. Ptolemy, and M. Rifai, “The Riddle of Protein Diagnostics: Future Bleak or Bright?” Clin. Chem., 59, pp 194-197 (2013).
M. Y. Liu, A. M. Xydadis, R, C, Hoogeveen, P. H. Jones, E. O. Smith, K. W. Nelson and C. M. Ballantyne, “Multiplexed Analysis of Biomarkers Related to Obesity and the Metabolic Syndrome in Human Plasma, Using the Luminex-100 System,” Clin. Chem., 51, pp 1102-1109 (2005).
A. X. Zhu, R. S. Finn, M. Mulcahy, J. Gurtler, W. Sun. J. D. Schwartz, R. P. Dalal, A. Joshi, R. R. Hozak, Y. Zhu, M. Ancukiewicz, R. K. Jain, F. Nugent, D. G. Duda, and K.
Stuart, “A Phase II and Biomarker Study of Ramucirumab, a Human Monoclonal Antibody Targeting the VEGF Receptor-2 in Patients with Advanced Hepatocellular Cancer,” Clin. Cancer Res., 19, pp 6614-6623 (2013).
Y. Hathout, E. Brody, R. R. Clemens, L. Cripe, R. K. DeLisle, P. Furlong, H Gordish-Dressman, L. Hache, E. Henricson, E. P. Hoffman, Y. M. Kobayashi, A. Lorts, J. K. Mah, C McDonald, B. Mehler, S. Nelson, M. Nikrad, B. Singer, F. Steele, D. Sterling, J. L. Sweeney, S. Williams and L. Gold, “Large-scale Serum Protein Biomarker Discovery in Duchenne Muscular Dystrophy,” PNAS, 112 pp 7153-7158 (2015).
N. Zolotarj ova, J. Martosella, G. Nicol, J. Bailey, B. E. Boyes, and W. C. Barrett, “Differences among techniques for high-abundant protein depletion,” Proteomics 5, pp. 3304-3313 (2005).
J. E. Bandow, “Comparison of protein enrichment strategies for proteome analysis of plasma,” Proteomics 10, pp. 1416-1425 (2010).
C. Tu, P. A. Rudnick, M. Y. Martinez, K. L. Cheek, S. E. Stein, R. J. C. Slebos and D. C. Lieber, “Depletion of Abundant Plasma Proteins and Limitations of Plasma Proteomics,” J. Prot. Res., 9, pp 4982-4991 (2010).
R. L. Gundry, Q. Fu, C. A. Jelinek, J. E. van Eyk, and R. J. Cotter, “Investigation of an albumin-enriched fraction of human serum and its albuminome,” Proteomics Clin Appl., 1, pp. 73-88 (2007).
R. J Holewinski, Z. J. Jin, M. P. Powell, M. D. Maust, and J. E. Van Eyk, “A Fast and Reproducible Method for Albumin Isolation and Depletion from Serum and Cerebrospinal Fluid,” Proteomics, 13, pp 743-750 (2013).
P. T. Van, V. Bass, D. Shiwarski, F. Lanni, and J. Minden, “High Dynamic Range Proteome Imaging with the Structured Illumination Gel Imager,” Electrophoresis, 35, pp 2642-55 (2014).
K. A. Dill, “Denatured States of Proteins,” Annu. Rev. Biochem., 60, pp 795-825 (1991).
Y. Moriyama and K. Takeda, “Re-formation of the Helical Structure of Human Serum Albumin by the Addition of Small Amounts of Sodium Dodecyl Sulfate after the Disruption of the Structure by Urea. A Comparison with Bovine Serum Albumin,” Langmuir, 15 pp 2003-2008 (1999).
Ellington, A. D.; Szostak, J. W. In vitro selection of RNA molecules that bind specific ligands. Nature 1990, 346, 818-822.
Tuerk, C.; Gold, L. Systematic evolution of ligands by exponential enrichment: RNA ligands to bacteriophage T4 DNA polymerase. Science 1990, 249, 505-510.
Sharma, A. K.; Heemstra, J. M. Small-molecule-dependent split aptamer ligation. J. Am. Chem. Soc. 2011, 133, 12426-12429.
Sharma, A. K.; Kent, A. D.; Heemstra, J. M. Enzyme-Linked Small-Molecule Detection using Split Aptamer Ligation. Anal. Chem. 2012, 84, 6104-6109.
Spiropulos, N. G.; Heemstra, J. M. Templating Effect in DNA Proximity Ligation Enables use of Non-Bioorthogonal Chemistry in Biological Fluids. Artif. DNA PNA XNA 2012, 3, 123-128.
Schmidt-Dannert, C.; Arnold, F. H. Directed evolution of industrial enzymes. Trends Biotechnol. 1999, 17, 135-136.
K.-A. Yang, R. Pei, D. Stefanovic, and M. N. Stojanovic, J. Am. Chem. Soc., 134, p. 4198, (2012).
Uphoff, K. W.; Bell, S. D.; Ellington, A. D. In vitro selection of aptamers: the dearth of pure reason. Curr. Opin. Struct. Biol. 1996, 6, 281-288.
McKeague, M.; DeRosa, M. C. Challenges and Opportunities for Small Molecule Aptamer Development. J. Nucleic Acids 2012, 2012, 748913.
G. P. Royer, F. A. Liberatore and G. M. Green, “Immobilization of Enzymes on Aldehydic Matrices by Reductive Alkylation,” Biochem. Biophys. Res. Comm, 64, pp 478-484 (1975).
L. D. Bowers and P. W. Carr, “Preparation and Characterization of Hexokinase Covalently Bound to Controlled Porosity Glass,” Biotechnol. Bioeng., 18, pp 1331-1334 (1976).
A. J. Muller and P. W. Carr, “Chromatographic Study of the Thermodynamic and Kinetic Characteristics of Silica-Bound Concanavalin A,” J. Chrom., 284, pp 33-51 (1984).
F. G. Helfferich, P. W. Carr, “Non-linear Waves in Chromatography: I. Waves, Shocks, and Shapes,” J. Chrom. A, 629 pp 97-122 (1993).
F. G. Helfferich, “Non-linear Waves in Chromatography II. Wave Interference and Coherence in Multicomponent Systems,” J. Chrom. A, 734, pp 7-47 (1996).
F. G. Helfferich, “Non-linear Waves in Chromatography III. Multicomponent Langmuir and Langmuir-like Systems,” J. Chrom. A, 768, pp 169-205 (1997).
G. Guiochon, Nonlinear and Preparative Chromatography, 2nd Ed., Elsevier (2006).
A. M. Petersen, F. M. Jahnke, and J. M. Heemstra, “Modulating the Substrate Selectivity of DNA Aptamers Using Surfactants,” Langmuir 2015, 31, 11769-11773.
Robertson, D. L.; Joyce, G. F. Nature 1990, 344, 467-468.
O'Sullivan, C. K. Anal. Bioanal. Chem. 2002, 372, 44-48.
Famulok, M.; Hartig, J. S.; Mayer, G. Chem. Rev. 2007, 107, 3715-3743.
Cho, E. J.; Lee, J.-W.; Ellington, A. D. Annu. Rev. Anal. Chem. 2009, 2, 241-264.
Mascini, M.; Palchetti, I.; Tombelli, S. Angew. Chem. Int. Ed. 2012, 51, 1316-1332.
Liu, J.; Cao, Z.; Lu, Y. Chem. Rev. 2009, 109, 1948-1998.
Bradbury, A.; Pluckthun, A. Nature 2015, 518, 27-9.
Otzen, D. Biochim. Biophys. Acta 2011, 1814, 562-591.
Neugebauer, J. M. In Methods in Enzymology, Murray, P. D., Ed.; Academic Press, 1990, pp 239-253.
Fendler, J. H.; Fendler, E. J. In Catalysis in Micellar and Macromoleular Systems, Fendler, J. H.; Fendler, E. J., Eds.; Academic Press, 1975, pp 230-253.
Paleologos, E. K.; Giokas, D. L.; Karayannis, M. I. Trends Anal. Chem. 2005, 24, 426-436.
Green, E.; Olah, M. J.; Abramova, T.; Williams, L. R.; Stefanovic, D.; Worgall, T.; Stojanovic, M. N. J. Am. Chem. Soc. 2006, 128, 15278-15282.
Stojanović, M. N.; Green, E. G.; Semova, S.; Nikić, D. B.; Landry, D. W. J. Am. Chem. Soc. 2003, 125, 6085-6089.
Reinstein, O.; Neves, M. A.; Saad, M.; Boodram, S. N.; Lombardo, S.; Beckham, S. A.; Brouwer, J.; Audette, G. F.; Groves, P.; Wilce, M. C.; Johnson, P. E. Biochemistry 2011, 50, 9368-76.
Yang, K.-A.; Pei, R.; Stefanovic, D.; Stojanovic, M. N. J. Am. Chem. Soc. 2012, 134, 1642-1647.
Feagin, T. A.; Olsen, D. P. V.; Headman, Z. C.; Heemstra, J. M. J. Am. Chem. Soc. 2015, 137, 4198-4206.
Nutiu, R.; Li, Y. J. Am. Chem. Soc 2003, 125, 4771-4778.
Burden, D. W. Random Primers 2012, 1-25.
Albright, L. M.; Slatko, B. E. Curr. Protoc. Nucleic Acid Chem. 2001, Appendix 3.
Jiwpanich, S.; Ryu, J.-H.; Bickerton, S.; Thayumanavan, S. J. Am. Chem. Soc. 2010, 132, 10683-10685.
Brown, D.; Lydon, J.; M., M.; Stuart-Tilley, A.; Tyszkowski, R.; Alper, S.
Histochem. Cell Biol. 1996, 105, 261-267.
Stoltenburg, R.; Reinemann, C.; Strehlitz, B., FluMag-SELEX as an advantageous method for DNA aptamer selection. Anal Bioanal Chem 2005, 383 (1), 83-91.
Gudiksen, K. L.; Gitlin, I.; Whitesides, G. M., Differentiation of proteins based on characteristic patterns of association and denaturation in solutions of SDS. Proceedings of the National Academy of Sciences of the U.S. Pat. No. 2,006,103 (21), 7968-7972.

Various features and advantages of the invention are set forth in the following claims.

1. A method for selecting a nucleic acid aptamer, the method comprising
providing a solution comprising a plurality of nucleic acids, a target molecule and a surfactant, whereupon at least one nucleic acid binds to the target molecule to form a complex,
separating the complex from the solution, and
separating the at least one nucleic acid from the complex,
wherein the at least one nucleic acid is the nucleic acid aptamer.

2. The method of claim 1, wherein the solution further comprises a reductant.

3. The method of claim 2, wherein the reductant is DTT, TCEP or a combination thereof.

4. The method of any of the preceding claims, further comprising the step of heating the solution.

5. The method of any of the preceding claims, wherein the target molecule comprises a small molecule, peptide or protein.

6. The method of any of the preceding claims, wherein the target molecule comprises at least one of HSA, an immunoglobulin, or a steroid.

7. The method of any of the preceding claims, wherein the surfactant comprises at least one of a non-ionic, zwitterionic or anionic surfactant.

8. The method of any of the preceding claims, wherein the surfactant is at least one of SDS, Triton X-100, Tween or Empigen BB.

9. The method of any of the preceding claims, wherein the surfactant is present in an amount of at least 0.1% (w/v) of the solution.

10. The method of any of the preceding claims, wherein the surfactant is present in an amount of at least 1% (w/v) of the solution.

11. The method of any of the preceding claims, wherein the surfactant is present in an amount of at least 4% (w/v) of the solution.

12. The method of any of the preceding claims, wherein the nucleic acid aptamer comprises RNA, DNA or any combination thereof.

13. The method of claim 2, wherein the nucleic acid aptamer comprises RNA or DNA, the target molecule comprises HSA, the surfactant comprises SDS, Empigen, Triton X-100 or Tween, and the reductant comprises DTT or TCEP.

14. A method for separating a target molecule from a sample, the method comprising
contacting the sample with a nucleic acid aptamer selected against a target molecule and a surfactant, whereupon the nucleic acid aptamer forms a complex with the target molecule, and
separating the complex from the sample.

15. The method of claim 14, wherein the surfactant is the same surfactant used in the selection of the nucleic acid aptamer according to the method of claim 1.

16. The method of any of claims 14-15, wherein the solution further comprises a reductant.

17. The method of claim 16, wherein the reductant is DTT, TCEP or a combination thereof.

18. The method of any of claims 14-17, wherein the target molecule comprises a small molecule or protein.

19. The method of any of any of claims 14-18, wherein the target molecule comprises at least one of HSA, an immunoglobulin, a peptide or a steroid.

20. The method of any of claims 14-19, wherein the surfactant comprises at least one of a non-ionic, zwitterionic or anionic surfactant.

21. The method of any of claims 14-20, wherein the surfactant is at least one of SDS, Triton X-100, Tween or Empigen BB.

22. The method of any of claims 14-21, wherein the surfactant is present in an amount of at least 0.1% (w/v) of the solution.

23. The method of any of claims 14-22, wherein the surfactant is present in an amount of at least 1% (w/v) of the solution.

24. The method of any of claims 14-23, wherein the surfactant is present in an amount of at least 4% (w/v) of the solution.

25. The method of any of claims 14-24, wherein the nucleic acid aptamer comprises RNA, DNA or any combination thereof.

26. The method of any of claims 14-25, wherein the nucleic acid aptamer is attached to a solid support.

27. The method of claim 26, wherein the solid support is a bead, a capillary tube, or the walls opened within a microfluidic device.

28. The method of claim 26, wherein the solid support is formed of a material comprising a silica gel, CPG, quartz, fused silica, a polymer, agarose, or any combination thereof.

29. The method of any of claims 26-28, wherein the nucleic acid aptamer is attached to the solid support via an amide linkage, a NHS linkage, a thiol linkage, a maleimide linkage, an azide linkage, an epoxide linkage, or any combination thereof.

30. The method of any of claims 14-28, further comprising separating the target molecule from the complex.

31. The method of claim 16, wherein the nucleic acid aptamer comprises RNA or DNA, the target molecule comprises HSA, the surfactant comprises SDS, Empigen or Tween, and the reductant comprises DTT or TCEP.

32. A method for purification of a biological sample, the method comprising
contacting the sample with a plurality of nucleic acid aptamers selected according to the method of claim 1 and a surfactant, whereupon at least one of the nucleic acid aptamers forms a complex with a target molecule, and
separating the complex from the biological sample.

33. The method of claim 32, wherein the surfactant is the same surfactant used in the selection of the nucleic acid aptamer according to the method of claim 1.

34. The method of any of claims 32-33, wherein the solution further comprises a reductant.

35. The method of claim 34, wherein the reductant is DTT, TCEP or a combination thereof.

36. The method of any of claims 32-35, wherein the target molecule is a small molecule, a peptide or a protein.

37. The method of any of any of claims 32-36, wherein the target molecule is at least one of HSA, an immunoglobulin, or a steroid.

38. The method of any of claims 32-37, wherein the surfactant comprises at least one of a non-ionic, zwitterionic or anionic surfactant.

39. The method of any of claims 32-38, wherein the surfactant is at least one of SDS, Triton X-100, Tween 80, Tween 20 or Empigen BB.

40. The method of any of claims 32-39, wherein the surfactant is present in an amount of at least 0.1% (w/v) of the solution.

41. The method of any of claims 32-40, wherein the surfactant is present in an amount of at least 1% (w/v) of the solution.

42. The method of any of claims 32-41, wherein the surfactant is present in an amount of at least 4% (w/v) of the solution.

43. The method of any of claims 32-42, wherein the nucleic acid aptamer comprises RNA, DNA or any combination thereof.

44. The method of any of claims 32-43, wherein the nucleic acid aptamer is attached to a solid support.

45. The method of claim 44, wherein the solid support is a bead, a capillary tube or the walls opened within a microfluidic device.

46. The method of claim 44, wherein the solid support is formed of a material comprising silica gel, CPG, quartz, fused silica, a polymer, agarose or any combination thereof.

47. The method of any of claims 44-46, wherein the nucleic acid aptamer is attached to the solid support via an amide linkage, a NHS linkage, a thiol linkage, a maleimide linkage, an azide linkage, an epoxide linkage, or any combination thereof.

48. The method of any of claims 44-47, wherein the solid support comprises a mixed bed or cartridge column.

49. The method of any of claims 32-48, wherein the biological sample is at least one of blood, plasma, serum, CSF, pleural effusion fluid, saliva, tears, urine, a cell lysate or a tissue extract.

50. The method of any of claims 32-49, further comprising separating the target molecule from the complex.

51. The method of claim 34, wherein the biological sample comprises human plasma, the nucleic acid aptamer comprises RNA or DNA, the target molecule comprises HSA, the surfactant comprises SDS or Tween, and the reductant comprises DTT or TCEP.
